(19) United States
(12) Patent Application Publication
(10) Pub. No.: US 2024/0115735 A1
(43) Pub. Date: Apr. 11, 2024

(54) MATERIALS AND METHODS FOR THE TREATMENT OF LYSOSOMAL ACID LIPASE DEFICIENCY (LAL-D)

(71) Applicant: RESEARCH INSTITUTE AT NATIONWIDE CHILDREN'S HOSPITAL, Columbus, OH (US)

(72) Inventor: Paul Taylor Martin, Bexley, OH (US)

(21) Appl. No.: 18/273,643

(22) PCT Filed: Jan. 26, 2022
(86) PCT No.: PCT/US22/13847

§ 371 Date: Jul. 21, 2023

Related U.S. Application Data
(60) Provisional application No. 63/142,257, filed on Jan. 27, 2021.

(51) Int. Cl. A61K 48/00; A61K 9/00; A61K 45/06; A61P 1/16; A61P 3/06; C12N 9/20; C12N 15/86

(52) U.S. Cl. CPC A61K 48/005; A61K 9/0019; A61K 45/06; A61P 1/16; A61P 3/06; C12N 9/20; C12N 15/86; C12N 2750/14143; C12Y 301/01013

(57) ABSTRACT

The disclosure provides gene therapy vectors, such as adeno-associated virus (AAV), designed for treatment of Lysosomal Acid Lipase Deficiency (LAL-D) disorders such as Wolman Disease and cholesterol ester storage disease (CESD). The disclosed rAAV provide a wild type lipase A (LIRA) cDNA to a subject in need which results in expression of the wild type protein.

This application contains, as a separate part of the disclosure, a Sequence Listing in computer-readable form which is incorporated by reference in its entirety and identified as follows: 54743_Seqlisting.txt; Size: 14,266 bytes; Created: Jan. 21, 2022.

### FIELD OF THE INVENTION

The disclosure provides gene therapy vectors, such as adeno-associated virus (AAV), designed for treatment of Lysosomal Acid Lipase Deficiency (LAL-D) disorders such as Wolman Disease and cholesterol ester storage disease (CESD). The disclosed rAAV provide a wild type human lipase A (LIPA) cDNA to a subject in need which results in expression of the wild type human LAL protein.

### BACKGROUND

Lysosomal Acid Lipase Deficiency, LAL-D, is a lysosomal storage disorder caused by recessive mutations in the Lipase A (LIPA) gene that result in a failure of the lysosomal acid lipase (LAL) protein to sufficiently hydrolyze cholesterol esters into free cholesterol and triglycerides into free fatty acids in the lysosome. LAL occupies a critical and essential position in the control of plasma lipoprotein levels and in the prevention of cellular lipid overload, especially in the liver and spleen (Li et al., Arterioscler Thromb Vasc Biol 139: 850-856, 2019; Aguisanda et al. Curr Chem Genom Transl Med 11: 1-18, 2017). The LIPA gene is the only gene with this lysosomal function in the human genome. LAL-D is a rare genetic disease, with prevalence ranging from 1 in 40,000 to 1 in 300,000, though disease incidence may be underestimated through failed diagnosis in some instances (Pastores et al., Lysosomal Acid Lipase Deficiency: Therapeutic Options. Drug Des Devel Ther 14: 591-601, 2020).

Null LIPA gene mutations cause Wolman disease (WD), a fatal disease of infancy named after Moshe Wolman, who reported one of the first cases (Abromov et al., AMA J Dis Child 91: 282-286, 1956). WD is characterized by hepatomegaly with liver dysfunction, dyslipidemia (elevated serum triglycerides and LDL-cholesterol with reduced HDL-cholesterol), hepatosplenomegaly, pulmonary fibrosis, and adrenal calcification and insufficiency. Infants manifest disease in the first month of life and fail to thrive, most likely due to liver disease combined with a failure to absorb nutrients through the intestinal lining. Median lifespan of untreated WD infants is 3.7 months. Partial loss of function LIPA mutations, usually with 1-12% of normal activity, give rise to cholesterol-ester storage disease (CESD), a later onset, less severe disease form. While CESD need not result in premature death, it is associated with significant morbidity, including liver fibrosis and cirrhosis (and also liver failure). Chronic dyslipidemia in LAL-D may also cause accelerated atherosclerosis and high risk of cardiac disease, including myocardial infarction, and cerebrovascular complications, including stroke. Liver biopsy in LAL-D patients typically demonstrate micro- and macro-vascular steatosis involving Kuppfer cells and hepatocytes, accompanied by fibrosis and cirrhosis as the disease progresses. Unlike other lysosomal storage disorders such as Gaucher disease and Niemann-Pick disease, there appears to be no primary CNS involvement (though histological studies are lacking).

While LAL-D is a rare genetic disorder, the pathology findings in LAL-D speak to larger and far more common significant health issues that are found in the general population. For example, reduced LAL-D activity is a biomarker for non-alcoholic fatty liver disease, a disorder affecting many millions of American adults and children. Such reductions in LAL activity are progressive with the increasing severity of liver disease; LIPA enzyme activity decreases from simple liver steatosis to non-alcoholic steatohepatitis to cryptogenic liver cirrhosis. Additionally, there is a LIPA haplotype strongly associated with coronary artery disease [10, 11]. The buildup of fatty acids in LAL-D mimics human conditions such as morbid obesity and obesity related to type II diabetes. Given how easily AAV transduces the liver, and how important the liver is in controlling lipoprotein-based metabolism, it is conceivable that LIPA gene therapy could be applied to these other genetic, and even non-genetic, human diseases related to obesity, based on the relationship of LAL-D to fat absorption in these other disorders.

### SUMMARY

The disclosure provides for a clinical AAV vector used in gene replacement therapy for LAL-D including those disorders caused by mutations in the LIPA gene and non-genetic disorders that are associated with lipid accumulation and storage.

In one aspect, described herein is a polynucleotide comprising (a) one or more regulatory control elements and (b) LIPA cDNA sequence. In some embodiments, the regulatory control element is a miniCMV promoter comprising a nucleotide sequence set forth in SEQ ID NO: 3, or fragments thereof which retain regulatory control or promoter activity. In some embodiments, the vector comprises a late SV40 poly adenylation sequence having the nucleotide sequence of SEQ ID NO: 5. In some embodiments, the LIPA cDNA is the LIPA variant 1 cDNA, and the LIPA cDNA comprises the polynucleotide sequence set forth in SEQ ID NO: 1.

In one embodiment, the disclosure provides for a rAAV comprising a nucleotide sequence that encodes a functional lysosomal acid lipase (LAL) protein, wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 1, wherein the protein retains LAL activity, such as the activity to hydrolyze cholesterol esters into free cholesterol and triglycerides into free fatty acids in the lysosome. For example, the nucleotide sequence that encodes a functional LAL protein may comprise one or more base pair substitutions, deletions or insertions which do affect the function of the LIPA protein. Furthermore, the nucleotide sequence that encodes a functional LIPA protein may comprise one or more base pair substitutions, deletions or insertions may increase or reduce expression of the LAL protein, and this change in expression pattern may be desired for treatment of the LAL-D or the disorder related to lipid storage and accumulation.

In another embodiment, the disclosure provides for a rAAV comprising a nucleotide sequence that encodes a functional LAL protein, wherein the protein comprises an amino acid sequence that has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 2, wherein the protein retains LAL activity, such as the activity to hydrolyze cholesterol esters into free cholesterol and triglycerides into fatty acids in the lysosome. For example, the nucleotide sequence that encodes a functional LAL protein may comprise one or more amino acid substitutions, deletions or insertions which do affect the function of the LAL protein.

The term “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. The percent identity of the sequences can be determined by techniques known in the art. For example, homology can be determined by a direct comparison of the sequence information between two polypeptide molecules by aligning the sequence information and using readily available computer programs such as ALIGN, ClustalW2 and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

In another aspect, the disclosure provides for an rAAV construct contained in the plasmid comprising the nucleotide sequence of SEQ ID NO: 4. For example, the rscrAAVrh74.miniCMV.LIPA vector comprises the nucleotide sequence within and inclusive of the ITR's of SEQ ID NO: 4. The rAAV vector comprises the 5′ ITR, miniCMV promoter, the coding sequence for the human LIPA gene, SV40 late polyA, and 3′ ITR. In this embodiment, the 3′ITR contains a deletion of the terminal resolution site (dTR), which inhibits Rep protein nicking of the single stranded viral genome. The presence of the dTR in the 3′ ITR increases self-complementary binding of the viral genome to itself, which it may do because of its small (2.2 kB) size that allows for a double-stranded viral genome to be packaged within the viral capsid. The self-complementary nature of rscAAVrh74.mCMV.LIPA facilitates both the speed and the extent of gene expression relative to constructs that remain as a single-stranded viral genome. In one embodiment, the vector comprises nucleotides 1853-3906 of SEQ ID NO: 4. The nucleotides within the ITRs may be in forward or reverse orientation. For example, the miniCMV promoter sequence, human LIPA gene sequence, and SV40 late polyA sequence and may be in forward or reverse orientation. In another embodiment, the vector comprises a nucleotide sequence that has about at least 85%, 86%, 87%, 88%, 89%, 90%, 91%, 92%, 93%, 94% 95%, 96%, 97%, 98%, 99%, or 100% sequence identity to the nucleotides of 1-4397 of SEQ ID NO: 4. The plasmid set forth in SEQ ID NO 4 further comprises kanamycin resistance and an origin of replication.

In another aspect, described herein is a recombinant adeno-associated virus (rAAV) having a genome comprising a polynucleotide sequence described herein. In some embodiments, the rAAV is of the serotype AAV1, AAV2, AAV3, AAV4, AAV5, AAV6, AAV7, AAV8, AAV9, AAVRH10, AAVrh74, AAVrh, AAV11, AAV12, AAV13, or Anc80, AAV7m8 or their derivatives.

In some embodiments, the genome of the rAAV comprises a miniCMV promoter and LIPA cDNA. An exemplary genome comprises the miniCMV promoter, and the LIPA cDNA such as the rscrAAVrh74.miniCMV.LIPA, the rAAV set out as nucleotides 1853-3906 of SEQ ID NO: 4. The miniaturized CMV promoter allows for AAV packaging of the self-complementary double-stranded viral genome, which is not allowed with promoters that are of a larger size.

In another aspect, described herein is an rAAV particle comprising an rAAV described herein.

Compositions comprising any of the rAAV described herein or any of the viral particles described herein. The disclosed composition may be formulated for any means of delivery, such as direct injection into the cerebrospinal fluid, intracerebroventricular delivery, intrathecal delivery, intraperitoneal delivery, intraarterial delivery, or intravenous delivery.

In addition, the disclosed composition is formulated for intravenous delivery or intraperitoneal delivery and comprises a dose of rAAV or rAAV particles of about 1e13 vg/kg to about 2e14 vg/kg, e.g. 8×10¹³ vg/kg.

Methods of treating LAL-D or a disorder related to lipid storage or accumulation in a subject in need thereof comprising administering a polynucleotide, an rAAV or an rAAV particle described herein are specifically contemplated. In some embodiments, the methods further comprise administering an immunosuppressing agent prior to, after or simultaneously with the polynucleotide, rAAV or rAAV particle. The LAL-D includes a disorder or disease caused by a mutation in the LIPA gene, such as Wolman disease or cholesterol ester storage disease. The disorder related to lipid storage or accumulation include coronary artery disease, atherosclerosis, type II diabetes, obesity or non-alcoholic fatty liver disease.

The disclosure also provides for methods of treating dyslipidemia or hypercholesterolemia in a subject in need thereof comprising administering a polynucleotide, an rAAV or an rAAV particle described herein are specifically contemplated. In some embodiments, the methods further comprise administering an immunosuppressing agent prior to, after or simultaneously with the polynucleotide, rAAV or rAAV particle.

The disclosure also provides for method of decreasing triglycerides, cholesterol, and/or fatty acids in a subject in need thereof comprising administering a polynucleotide, an rAAV or an rAAV particle described herein are specifically contemplated. In some embodiments, the methods further comprise administering an immunosuppressing agent prior to, after or simultaneously with the polynucleotide, rAAV or rAAV particle.

In any of the disclosed methods, the polynucleotide, rAAV, rAAV particle or composition are intravenously delivered to the subject. In some embodiments, the method further comprises a step of administering an immunosuppressing agent. For example, the polynucleotide, rAAV, rAAV particle or composition is administered simultaneously, prior to or after administration of an immunosuppressing agent, such as prednisone, prednisolone, rapamycin, methotrexate, myophenolate mofetil, tacrolimus, mycophenolate, or rituximab. In any of the methods, the subject has a mutation in the LIPA gene. These mutations include those currently known, such as those set out in Table 1 herein, or a mutation(s) in the LIPA gene identified in the future that is associated with LAL-D.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). in some embodiments, the subject is a human. In some embodiments, the subject is a pediatric subject. In some embodiments, the subject is a pediatric subject, such as a subject ranging in age from 1 to 10 years or the subject is an infant ranging in age for one month to 12 months. In some embodiments, the subject is 4 to 15 years of age. The subject, in on embodiment, is an adolescent subject, such as a subject ranging in age from 10 to 19 years. In other embodiments, the subject is an adult (18 years or older).

In another aspect, described herein is the use of a polynucleotide, an rAAV or an rAAV particle described herein in the preparation of a medicament for the treatment of an LAL-D or a disorder related to lipid storage or accumulation. In some embodiments, the LAL-D is Wolman disease or cholesterol ester storage disease. In additional embodiments, the disorder related to lipid storage or accumulation is coronary artery disease, atherosclerosis, type II diabetes, obesity or non-alcoholic fatty liver disease.

In addition, described herein is the use of a polynucleotide, an rAAV or an rAAV particle described herein in the preparation of a medicament for the treatment dyslipidemia or hypercholesterolemia in a subject in need thereof.

The disclosure also provides for use of a polynucleotide, an rAAV or an rAAV particle described herein in the preparation of a medicament for decreasing triglycerides, cholesterol, and/or fatty acids in a subject in need thereof.

For example, any of the disclosed medicaments are formulated for intravenous or intraperitoneal delivery. In some embodiments, the medicament is administered simultaneously, prior to or after administration of an immunosuppressing agent, such as prednisone, prednisolone, rapamycin, methotrexate, myophenolate mofetil, tacrolimus, mycophenolate, or rituximab.

In another aspect, described herein is a composition comprising a polynucleotide, an rAAV, an rAAV particle or composition described herein for the treatment of LAL-D or a disorder related to lipid storage or accumulation. In some embodiments, the LAL-D is Wolman disease or cholesterol ester storage disease. In additional embodiments, the disorder related to lipid storage or accumulation is coronary artery disease, atherosclerosis, type II diabetes, obesity or non-alcoholic fatty liver disease.

In addition, described herein is a composition comprising a polynucleotide, an rAAV, an rAAV particle or composition described herein for the treatment dyslipidemia or hypercholesterolemia in a subject in need thereof.

In a further aspect, described herein is a composition comprising a polynucleotide, an rAAV, an rAAV particle or composition described herein for decreasing triglycerides, cholesterol, and/or fatty acids in a subject in need thereof.

For example, any of the disclosed compositions are formulated for intravenous or intraperitoneal delivery. In some embodiments, the composition is administered simultaneously, prior to or after administration of an immunosuppressing agent. In another embodiment, the composition further comprises an immunosuppressing agent. Exemplary, immunosuppressing agents include prednisone, prednisolone, rapamycin, methotrexate, myophenolate mofetil, tacrolimus, mycophenolate, or rituximab.

### BRIEF DESCRIPTION OF THE FIGURES

FIG. 1 provides a schematic to the rscrAAVrh74.miniCMV.LIPA.

FIG. 2 demonstrates that serum measures liver enzymes in treated and untreated Lipa^{-/-} mice at 4 months of age. Alanine aminotransferase (ALT) and aspartate aminotransferase (AST) activity in serum from wild type (WT), mock-treated Lipa^{-/-}, and rAAVrh74.mCVM.LIPA-treated Lipa^{-/-}mice (treated with 8×10¹³ vg/kg AAV IV at postnatal day 1, analyzed at 4 mo). Errors are SEM for n=6(WT), 7 (Lipa^{-/-}) or 8 (Lipa^{-/-}Treated) mice/grp, with roughly even male and female representation. ***p<0.001, ****p<0.0001.

FIG. 3 demonstrates that serum LDL-cholesterol levels at 4 months of age. LDL-cholesterol level in serum from wild type (WT), mock-treated Lipa^{-/-}, and rAAVrh74.mCVM.LIPA-Treated Lipa^{-/-}mice (treated with 8×10¹³ vg/kg AAV IV at postnatal day 1, analyzed at 4 mo). Errors are SEM for n=6 (WT), 7 (Lipa^{-/-}) or 8 (Lipa^{-/-}Treated) mice/grp. **p<0.01, ***p<0.001.

FIG. 4 provides a comparison of body and organ weights in treated and untreated Lipa^{-/-} mice in comparison to wild type. (A) total body weight, (B) liver weight, (C) spleen weight, (D) intestine weight, (E) heart weight, (F) kidney weight, (G) brain weight, and (H) muscle weight were compared. Treated Lipa^{-/-} mice were given an IV dose of 8×10¹³ vg/kg rscAAVrh74.mCVM.LIPA at postnatal day 1 (P1) and analyzed at 6 months of age. Errors are SEM for n=4-5/grp. *p<0.05, **p<0.01, ***p<0.001. Note that while Lipa^{-/-}mice gain weight, this pales in comparison to the dramatic increases in organ weight for certain organs (liver and spleen). There are milder weight gains for other organs (kidney and heart), while there is no weight gain (brain) or reduced weight gain for others (e.g., muscle, most likely due to nutritional deficits from fat deposition in the intestine).

FIG. 5 provides a representative oil red/hematoxylin staining of organs from mock-treated and AAV-treated Lipa^{-/-} mice and wild type controls. Lipa^{-/-} Treated mice were given 8×10¹³ vg/kg rscAAVrh74.mCVM.LIPA gene therapy IV at postnatal day 1 and tissues were stained with Oil Red O, to identify lipid overload from disease, at 6 months of age. In all instances, Oil Red O staining was very reduced by treatment, though not absent. Bar is 200 μm.

FIG. 6 provides cellular triglyceride levels in liver and spleen after AAV treatment of Lipa^{-/-}mice. (A) Liver and (B) Spleen from 6-month-old wild type (WT), Lipa^{-/-}, and Lipa^{-/-}Treated mice (treated IV with 8×10¹³ vg/kg of rscAAVrh74.mCVM.LIPA at postnatal day 1). Errors are SEM for n=4-5/grp. *p<0.05, **p<0.001, ***p<0.001, ****p<0.0001.

FIG. 7 provides AAV vector genome biodistribution per nucleus. Injection if Lipa^{-/-} mice (LIPA KO) at 2 months led to increased transduction of liver lymph node, spleen and intestine relative to injection at P1 when assayed at 4 months of age. Errors are SD for n=3/group. Vgs were measured per ug of genomic DNA, then converted to vg/nucleus (or per cell).

FIG. 8 demonstrates LIPA gene expression induced by rscAAVrh74.mCMV.LIPA. Injection if Lipa^{-/-} mice (LIPA KO) at 2 months led to increased gene expression in liver lymph node, spleen and intestine relative to injection at P1 when assayed at 4 months of age. Errors are SD for n=3/group. Gene expression in treated LIPA KO mice is compared to normal gene expression in wild type mouse tissue.

FIG. 9 provides images of whole livers in 4 month old mice. LIPA KO mice, untreated, showed high fat content and increased size relative to wild type (FVBn). Treatment at P1 led to reduced size, but some fat content remained, while treatment at 2 mo removed all fat content.

FIG. 10 provides Liver cholesterol and triglyceride content after rscAAVrh74.mCMV.LIPA treatment in 4 mo LIPA KO mice compared to wild type (FVBn). Errors are SD.

FIG. 11 provides organ weights at 4 months after treatment with rscAAVrh74.mCMV.LIPA gene therapy. Errors are SD for n=2-5/grp.

FIG. 12 provides serum LIPA enzyme activity levels after rscAAVrh74.mCMV.LIPA gene therapy treatment at 4 months. Errors are SD

FIG. 13 provides liver LIPA enzyme activity after treatment with rscAAVrh74.mCMV.LIPA at 4 months of age. Errors are SD.

FIG. 14A-14H provide data demonstrating that rscAAVrh74.miniCMV.LIPA treatment reverses hepatosplenomegaly and elevated serum liver enzymes in Lipa^{-/-}mice. (A) Schematic of treatment plan of Lipa^{-/-}mice. (B) Gross pathology of liver and spleen from wild type, untreated Lipa^{-/-}and treated Lipa^{-/-}mice. Scale bar=1 cm. (C-F) Relative weights of liver, spleen, intestines, and mesenteric lymph node. (G-H) Serum alanine aminotransferase (ALT) and aspartate aminotransferase (AST) levels. All data represented as mean ±SD (n=5−8). Statistical significance between groups is denoted by different letters, P<0.05, using one-way ANOVA with Tukey's post-hoc test. Schematic created with BioRender.com. ITR=inverted terminal repeats; pA=SV40 polyA; dTlR=mutated ITR.

FIG. 15 provides Kaplan-Meier survival curve of untreated Lipa^{-/-}vs Lipa^{-/-}treated at P2. Treatment with gene therapy extends lifespan beyond the 224-day median survival.

FIG. 16A-16H demonstrates muscle atrophy may contribute to ambulation differences in Lipa^{-/-}mice. (A) Body weight of mice at 2, 4, 6 months. (B-C) Relative weight of gastrocnemius muscle and quadricep muscle at 6 months. (D-H) Open field analysis at 6 months. All data represented as mean ±SD (n=5−8). Statistical significance between groups is denoted by different letters, P<0.05, using one-way ANOVA with Tukey's post-hoc test.

FIG. 17A-17E demonstrates LIPA expression after rscAAVrh74.miniCMV.LIPA treatment leads to increased lysosomal acid lipase enzyme activity. (A) Biodistribution of AAV in various organs and tissues. Vector genomes (vg) per nucleus were quantified using quantitative real-time PCR. (B) Relative expression of AAV-introduced human LIPA, relative to the endogenous mouse Lipa, normalized to 18S mRNA. (C-E) Lysosomal acid lipase enzyme activity in liver, spleen, and serum. All data represented as mean ±SD (n=5−8). Statistical significance between groups is denoted by different letters, P<0.05, using one-way ANOVA with Tukey's post-hoc test.

FIGS. 183A-18J demonstrates that cholesterol and triglyceride content is reduced with treatment. (A-B) Cholesterol content in (A) liver and (B) spleen. (C-D) Triglyceride control in (C) liver and (D) spleen. (E-I) Serum lipid panel. (J) Oil Red O (ORO) staining of liver, spleen and intestines tissue sections. Neutral lipids are stained red with ORO. Tissue sections were counter-stained with hematoxylin (purple). Scale bar=25 μm. All data represented as mean ±SD (n=5−8). Statistical significance between groups is denoted by different letters, P<0.05, using one-way ANOVA with Tukey's post-hoc test.

FIG. 19 provides immunhistochemical staining of LIPA and CD68 in liver sections. LIPA immunostaining in treated livers (brown). Macrophages are stained with anti-CD68 (brown). Tissue sections were counter-stained with hematoxylin (purple). Scale bar=100 μm.

FIG. 20A-20E demonstrate that lower dose of rscAAVrh74.miniCMV.LIPA still show therapeutic benefits. (A) Gross pathology of the liver and spleen in the untreated WT and Lipa^{-/-}and treated Lipa^{-/} at different doses with rscAAVrh74.miniCMV.LIPA. Scale bar=1 cm. (B-E) Relative weight of liver (B), spleen (C), intestines (D), and lymph node (E) after treatment with different doses. (F-G) Serum ALT and AST levels with different doses. All data represented as mean ±SD (n=3−8). Statistical significance between groups is denoted by different letters, P<0.05, using one-way ANOVA with Tukey's post-hoc test.

FIGS. 21A-21E demonstrate that all doses of rscAAVrh74.miniCMV.LIPA treatment results in restored LIPA expression and lysosomal acid lipase enzyme activity. (A) Biodistribution of AAV decreases with decreasing dose in the liver, spleen, intestine, lymph node, heart, and lung. (B) LIPA expression also decreases with dose in the liver, spleen, intestine, lymph node, heart, and lung. (C-E) Lysosomal acid lipase activity in the liver, spleen, and serum. All data represented as mean ±SD (n=3−8). Statistical significance between groups is denoted by different letters, P<0.05, using one-way ANOVA with Tukey's post-hoc test.

FIGS. 22A-22D demonstrate that lipoid content is reduced with treatment at all doses. (A) Cholesterol levels in the liver. (B) Cholesterol content in the spleen. (C) Triglyceride content in the liver. (D) Triglyceride content in the spleen. All data represented as mean ±SD (n=3−8). Statistical significance between groups is denoted by different letters, P<0.05, using one-way ANOVA with Tukey's post-hoc test.

FIG. 23 provides an annotated sequence of ptrs-miniCMV.LIPA.KanR (5626 bp).

### DETAILED DESCRIPTION

While enzyme replacement protein therapy has shown clinical efficacy in WD and CESD patients and is approved by the FDA, such treatments require protein infusions every two weeks and give rise to only partial clinical correction (Burton et al. N Engl J Med 373: 1010-1020, 2015). In the phase 3 enzyme replacement clinical trial of Sebelipase Alfa (also called Kanuma), for example, elevations in serum aspartate aminotransferase (AST), alanine aminotransferase (ALT), total bilirubin, and low-density lipoprotein (LDL)-cholesterol, were, on average, reduced only by 50%, at best, with a 32% overall reduction in liver fat content and a 6.8% reduction in spleen volume (Burton et al. supra). As demonstrated herein, AAV delivery of the LIPA gene can have far greater impacts on these measures in a LAL-D mouse model.

The disclosure provides a recombinant (r) self-complementary (sc) AAV vector, rscAAVrh74.mCMV.LIPA, for use in treating WD and CESD patients. The rhesus 74 (rh74) serotype of AAV, originally isolated from the spleen of a rhesus macaque has shown safety at high intravenous doses (2−10¹⁴ vg/kg) in clinical trials with pediatric patients. rAAVrh74 is similar to rAAV8, rAAV9, and rAAVrh.10 in that it shows a high propensity to enter tissues after intravenous (IV) delivery to the blood, allowing for systemic multi-organ perfusion of the designed gene therapy using a single dose scheme (Zygmunt et al., Mol Ther Methods Clin Dev 15: 305-319, 2019). This dosing can last, in theory, at least in post-mitotic cells, for the lifetime of the animal (Chicoine et al., Mol Ther 22: 713-724, 2014; Martin et al., Am J Physiol Cell Physiol 296: C476-488, 2009). AAV is unique in its safety profile, as the viral genome, once transduced into its carrier cell, remains stably expressed as an episomal DNA and only very rarely ever integrates into the host genome (Grieger et al., Adv Biochem Eng Biotechnol 99: 119-145, 2005; Xiao et al. J Virol 72: 2224-2232, 1998). As with almost all AAV serotypes, liver and spleen are the most highly perfused organs when rAAVrh74 is delivered intravenously (Bish et al., Hum Gene Ther 19: 1359-1368, 2008) Liver and spleen typically receive logarithmically higher numbers of AAV DNA vector genomes (vg) than for other organs when adult animals are dosed, and this is true in multiple mammalian species, including humans, rhesus macaques, dogs, and rodents, including mice (Cunningham et al. Methods Mol Biol 1937: 213-219., 2019, Palaschak et al., Methods Mol Biol 1950: 333-360, 2019). Such features make AAV an ideal gene delivery method for treatment of genetic disorders such as LAL-D, where liver and spleen are the most affected organs (Aguisanda et al., supra; Burton et al. supra.).

The disclosed AAV vector is optimized for therapeutic usefulness in LAL-D. The self-complementary (sc) technology allows for binding of the single-stranded viral DNA genome onto itself, thereby priming second strand DNA synthesis. This self-complimentary element both quickens and strengthens gene expression relative to constructs lacking the self-complimentary element. Use of the self-complimentary technology is important for effective treatment of LAL-D, as children with complete deficiency become severely ill within a week or two after birth. Thus, use of a single-stranded rAAV vector, which will take 3-4 week for maximal onset of gene expression, would not be ideal for preventing a disease with such an early and severe onset. Because only about 2.2 kB of DNA can be packaged into self-complementary AAV vectors, a little less than half what can normally be packaged in a single-stranded vector (4.7 kB), a miniaturized Cytomegalovirus (mCMV) promoter was used to allow for all of the required DNA elements to be included along with the full-length human LIPA transgene. This miniaturized mCMV promoter has been used by Fu and McCarty to drive scAAV vectors to treat other lysosomal storage disorders such as Mucopolysaccharidosis, where the mCMV promoter shows the ability to induce gene expression across a broad spectrum of cells and tissues II (Fu et al., Mol Ther Methods Clin Dev 10: 327-340, 2008). The size limitation of AAV in the self-complimentary form, coupled with the size of the LIPA gene itself, make it impossible to use a full-length form of the CMV promoter to produce a self-complementary AAV form containing the LIPA gene. AAV yields were reduced 19-fold when the full-length CMV promoter was used, likely from reduced genomic packaging into viral capsids because of the increased size of the AAV genome. Packaging yields from a standard small scale AAV preparation average 1.15±0.15×10¹³ vg when the miniCMV is used and average only 6.0±1.1×10¹¹ vg when the full-length CMV promoter is used (n=2/group). Thus, miniCMV is an important design element of this AAV vector.

### LIPA Mutations

The LIPA gene is located on human chromosome 10q23.2-23.3 and consists of 10 exons spread over approximately 38 kb. LIPA has 3 transcript variants: Variant 2 (NM_000235) lacks an internal segment in the 5′ UTR compared with variant 1 (NM_001127605). The two variants encode the same protein isoform in size of 399 amino acids (AAs), which has been experimentally validated by cDNA cloning (Baratta et al., World J Gastroenterol 25: 4172-4180). The annotated variant 3 (NM_001288979) lacks two consecutive exons in the 5′ region, which results in translation initiation at a downstream AUG and presumably a shorter protein isoform consists of 283 AAs. (Li and Zhang, Arterioscler Thromb Vasc Biol. 39(5): 850-856, 2019).

There are at least 59 known mutations in the LIPA gene. Examples of these mutations are provided in Table 1 below.

| DNA | Protein | Citation |
|---|---|---|
| | p.T16P | Morris et al. |
| | | Arterioscler Thromb |
| | | Vasc Biol. 2017 June; |
| | | 37(6): 1050-1057. |
| | p.T16W | Morris et al. |
| | | Arterioscler Thromb |
| | | Vasc Biol. 2017 June; |
| | | 37(6): 1050-1057. |
| | p.(Trp140*) | |
| | p.(Arg218*) | |
| | p.(Gly266*), | |
| c.67G > A | p.Gly23Arg | |
| c.129C > G | p.Tyr43* | Ries et al Hum |
| | | Mutat, 12 (1998) |
| | | pp. 44-51 |
| c.253C > A | p.Gln85Lys | |
| c.260G > T; | p.Gly87Val | |
| c.294C > G; | p.Asn98Lys | |
| c.229 + 3A > C | | Pisciotta et al. |
| | | Arterosclerosis |
| | | 265: 124-132, 2017 |
| c.386A > G | p.His129Arg | Consuelo-Sanchez |
| | | Ann. Hepatol. 18(4): |
| | | 646-650, 2019 |
| c.414dup; | p.Phe139Ilefs*7 | Mayatepek et al. |
| | | Hum |
| | | Mutat, 12 (1998), |
| | | pp. 44-51 |
| | p.(Thr288Ile), | |
| | p.(Leu294Ser) | |
| c.822 + 37A > G | | |
| c.894G > A | p.Gln298Gln | Mutoni et al. Hum |
| | | Genet 95: 491-494, |
| | | 1995 |
| c.894G > A | p.delS275_Q298 | Consuelo-Sanchez |
| | | Ann. Hepatol. 18(4): |
| | | 646-650, 2019 |
| c.1024G > A; | p.Gly342Arg | Consuelo-Sanchez |
| | | Ann. Hepatol. 18(4): |
| | | 646-650, 2019 |
| | p.(Asp345Asn), | Pisciotta et al. |
| | | Arterosclerosis |
| | | 265: 124-132, 2017 |

### AAV Gene Therapy

The present disclosure provides for gene therapy vectors, e.g. rAAV vectors expressing the LIPA cDNA, and methods of treating Lysosomal Acid Lipase Deficiency (LAL-D), including Wolman disease and cholesterol ester storage disease. The disclosed gene therapy vectors are useful for treating disorders related to lipid storage and accumulation such as coronary artery disease such as atherosclerosis, type II diabetes, obesity and non-alcoholic fatty liver disease. In addition, the disclosed gene therapy vectors are useful for decreasing triglycerides, cholesterol, and/or fatty acids in a subject in need thereof, and for treating dyslipidemia or hypercholesterolemia in a subject in need thereof.

As used herein, the term “AAV” is a standard abbreviation for adeno-associated virus. Adeno-associated virus is a single-stranded DNA parvovirus that grows only in cells in which certain functions are provided by a co-infecting helper virus. There are currently at least thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including an inverted terminal repeat (ITRs). Exemplary ITR sequences may be 130 base pairs in length or 141 base pairs in length, such as the ITR sequence set out in SEQ ID NOS: 6 and 7. The nucleotide SEQ ID NO: 6 is an exemplary 5′ ITR, while the nucleotide sequence of SEQ ID NO: 7 is an exemplary 3′ ITR, which contains a deletion of the terminal resolution site (referred to as “dTR”). Deletion of the terminal resolution site inhibits Rep protein nicking of the single stranded viral genome. The presence of the dTR in the 3′ ITR increases self-complementary binding of the viral genome to itself, which it may do because of its small (2.2 kB) size that allows for a double-stranded viral genome to be packaged within the viral capsid.

There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes (e.g., AAV1, AAV2, AAV3, AAV4, AAV5, AAV6, AAV7, AAV8, AAV9, AAVRH10, AAVRH74, AAV11, AAV12, AAV13, or Anc80, AAV7m8 and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art.

The provided recombinant AAV (i.e., infectious encapsidated rAAV particles) comprise a rAAV genome. The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.” In exemplary embodiments, the genomes of both rAAV lack AAV rep and cap DNA, that is, there is no AAV rep or cap DNA between the ITRs of the genomes.

The rAAV genomes provided herein, in some embodiments, comprise one or more AAV ITRs flanking the transgene polynucleotide sequence. The transgene polynucleotide sequence is operatively linked to transcriptional control elements (including, but not limited to, promoters, enhancers and/or polyadenylation signal sequences) that are functional in target cells to form a gene cassette. Examples of promoters are the miniCMV promoter having the nucleotide sequence of SEQ ID NO: 3. Additional promoters are contemplated herein including, but not limited to the chicken β actin promoter (CBA), the P546 promoter the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

Additionally provided herein are a miniCMV promoter sequence, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of the miniCMV (SEQ ID NO: 3) sequence which exhibit transcription promoting activity.

Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

rAAV genomes provided herein comprises a polynucleotide (SEQ ID NO: 1) encoding LIPA protein. In some embodiments, the rAAV genomes provided herein comprises a polynucleotide that encodes a polypeptide comprising an amino acid sequence that is at least: 90%, 91%, 92%, 93%, 94%, 95%, 96%, 97%, 98%, or 99% identical to the amino acid sequence encoded by the LIPA cDNA (SEQ ID NO 1).

rAAV genomes provided herein comprises a nucleotides 1853-3906 of SEQ ID NO: 4. In some embodiments, the rAAV genomes provided herein comprises a polynucleotide that at least: 90%, 91%, 92%, 93%, 94%, 95%, 96%, 97%, 98%, or 99% identical to the nucleotide sequences of nucleotides 1853-3906 of SEQ ID NO: 4.

rAAV genomes provided herein, in some embodiments, a polynucleotide sequence that encodes an LAL protein and that hybridizes under stringent conditions to the polynucleotide sequence set forth in SEQ ID NO: 1 or the complement thereof.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-9, AAV-1, AAV-2, AAV-3, AAV-4, AAV-5, AAV-6, AAV-7, AAVrh.74, AAV-8, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658.776 ; WO 95/13392; WO 96/17947; PCT/U598/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG).

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10⁷, 1×10⁸, 1×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 7×10¹³, about 8×10¹³, about 9×10¹³, about 1×10¹⁴, about 2×10¹⁴, about 3×10¹⁴, about 4×10¹⁴, about 5×10¹⁴, about 1×10¹⁵, to about 1×10¹⁸, or more total viral genomes.

Dosages of about 1×10⁹ to about 1×10¹⁰, about 5×10⁹ to about 5×10¹⁰, about 1×10₁₀ to about 1×10¹¹, about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, about 1×10¹³ to about 1×10¹⁵ vg and about 6×10¹³ to about 1.0×10¹⁴ vg are also contemplated. One dose exemplified herein is 1×10¹³ vg administered via intravenous or intraperitoneal delivery.

Dosages are also may be expressed in units of vg/kg. Dosages contemplated herein include about 1×10⁷ vg/kg, 1×10⁸ vg/kg, 1×10⁹ vg/kg, 5×10⁹ vg/kg, 6×10⁹ vg/kg, 7×10⁹ vg/kg, 8×10⁹ vg/kg, 9×10⁹ vg/kg, 1×10¹⁰ vg/kg, 2×10¹⁰ vg/kg ¹⁰, 3×10¹⁰ vg/kg, 4×10¹⁰ vg/kg, 5×10¹⁰ vg/kg, 1×10¹¹ vg/kg, about 1×10¹² vg/kg, about 1×10¹³ vg/kg, about 1.1×10¹³ vg/kg, about 1.2×10¹³ vg/kg, about 1.3×10¹³ vg/kg, about 1.5×10¹³ vg/kg, about 2×10¹³ vg/kg, about 2.5×10¹³ vg/kg, about 3×10¹³ vg/kg, about 3.5×10¹³ vg/kg, about 4×10¹³ vg/kg, about 4.5×10¹³ vg/kg, about 5×10¹³ vg/kg, about 6×10¹³ vg/kg, about 7×10¹³, about 8×10¹³, about 9×10¹³, about 1×10¹⁴ vg/kg, about 2×10¹⁴ vg/kg, about 3×10¹⁴ vg/kg, about 4×10¹⁴ vg/kg about 5×10¹⁴ vg/kg, about 1×10¹⁵ vg/kg, to about 1×10¹⁸ vg/kg.

Dosages of about 1×10⁹ vg/kg to about 1×10¹⁰ vg/kg, about 5×10⁹ vg/kg to about 5×10¹⁰ vg/kg, about 1×10¹⁰ vg/kg to about 1×10¹¹ vg/kg, about 1×10¹¹ vg/kg to about 1×10¹⁵ vg/kg, about 1×10¹² vg/kg to about 1×10¹⁵ vg/kg, about 1×10¹² vg/kg to about 1×10¹⁴ vg/kg, about 1×10¹³ vg/kg to about 2×10¹⁴ vg/kg, about 1×10¹³ vg/kg to about 1×10¹⁵ vg/kg and about 6×10¹³ vg/kg to about 1.0×10¹⁴ vg/kg are also contemplated. One dose exemplified herein is 1×10¹³ vg/g administered via intravenous or intraperitoneal delivery.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Example of a LAL-D contemplated for prevention or treatment with methods of the disclosure is Wolman Disease and Cholesterol Ester Storage Disease or a disorder related to lipid storage or accumulation such as coronary artery disease, atherosclerosis, type II diabetes, obesity, r non-alcoholic fatty liver disease, dyslipidemia or hypercholesterolemia.

Combination therapies are also contemplated by the disclosure. Combination as used herein includes both simultaneous treatment and sequential treatments. Combinations of methods of the disclosure with standard medical treatments are specifically contemplated, as are combinations with novel therapies. In some embodiments, the combination therapy comprises administering an immunosuppressing agent in combination with the gene therapy disclosed herein.

Administration of an effective dose of the compositions may be by routes standard in the art including, but not limited to, intramuscular, parenteral, intravenous, intraarterial, intraperitoneal, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the wild type LAL protein.

The disclosure provides for local administration and systemic administration of an effective dose of rAAV and compositions of the disclosure. For example, systemic administration is administration into the circulatory system so that the entire body is affected. Systemic administration includes enteral administration such as absorption through the gastrointestinal tract and parenteral administration through injection, infusion or implantation.

Transduction of cells with rAAV of the disclosure results in sustained expression of the LAL protein. The present disclosure thus provides methods of administering/delivering rAAV which express LAL protein to an animal, preferably a human being. These methods include transducing cells with one or more rAAV of the present disclosure.

The term “transduction” is used to refer to the administration/delivery of the coding region of the LIPA gene to a recipient cell either in vivo or in vitro, via a replication-deficient rAAV of the disclosure resulting in expression of LAL the recipient cell.

### Immunosuppressing Agents

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins and polypeptides, and di peptide boronic acid molecules, such as Bortezomib.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, methotrexate, hydrocortisone, methylprednisolone, deflazacort, budesonide or prednisone.

Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include rapamycin (also known as sirolimus), everolimus, and temsirolimus.

The immunosuppressing agents include immune suppressing macrolides. The term “immune suppressing macrolides” refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and rapamycin (also known as sirolimus).

Purine analogs block nucleotide synthesis and include IMDH inhibitors. Exemplary purine analogs include azathioprine, mycophenolate such as mycophenolate acid or mycophenolate mofetil and lefunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immuosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

The following EXAMPLES are provided by way of illustration and not limitation. Described numerical ranges are inclusive of each integer value within each range and inclusive of the lowest and highest stated integer.

### EXAMPLES

### Example 1

### Gene Therapy Constructs Encoding LIPA

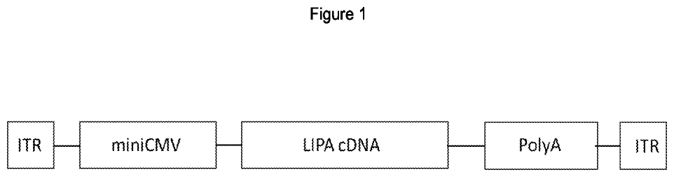

AAV genome constructs encoding LIPA were generated as set forth in FIG. 1, which depicts the AAVrh74 vector design with the full-length transcript of LIPA cDNA under the control of a miniCMV promoter (SEQ ID NO: 3).

A human GFP cDNA clone was obtained from Origene, Rockville, MD. The LIPA cDNA alone was further subcloned into a self-complementary AAVrh74 genome under the control of a miniCMV promoter. The plasmid construct also included an intron such as the simian virus 40 (SV40) chimeric intron, and a polyadenylation signal (PolyA). The constructs were packaged into either AAVrh74 genome.

The LIPA cDNA expression cassette had a Kanamycin resistance gene, and an optimized Kozak sequence, which allows for more robust transcription. rAAV vectors were produced by a modified cross-packaging approach whereby the AAVrh74 vector genome can be packaged into multiple AAV capsid serotypes [Rabinowitz et al., J Virol. 76 (2):791-801 (2002)]. Production was accomplished using a standard three plasmid DNA/CaPO4 precipitation method using HEK293 cells. HEK293 cells were maintained in DMEM supplemented with 10% fetal bovine serum (FBS) and penicillin and streptomycin. The production plasmids were: (i) plasmids encoding the therapeutic proteins, (ii) rep2-capX modified AAV helper plasmids encoding cap serotype AAVrh74 isolate, and (iii) an adenovirus type 5 helper plasmid (pAdhelper) expressing adenovirus E2A, E4 ORF6, and VA I/II RNA genes. A quantitative PCR-based titration method was used to determine an encapsidated vector genome (vg) titer utilizing a Prism 7500 Taqman detector system (PE Applied Biosystems). [Clark et al., Hum Gene Ther. 10 (6): 1031-1039 (1999)]. A final titer (vg ml-1) was determined by quantitative reverse transcriptase PCR using the specific primers and probes utilizing a Prism 7500 Real-time detector system (PE Applied Biosystems, Grand Island, NY, USA). Aliquoted viruses were kept at −80° C.

All plasmids used to make AAV genomes to be packaged also contain a Kanamycin resistance gene (KanR) outside of the ITR sequences used for packaging of the genome. This allows for the DNA encoding the AAV genome to be transformed into bacteria to produce large amounts of DNA in the presence of Kanamycin, which will kill all non-transformed bacteria. KanR is not packaged into the AAV capsid in the AAV genome used to treat patients, but its presence allows for DNA production in bacteria.

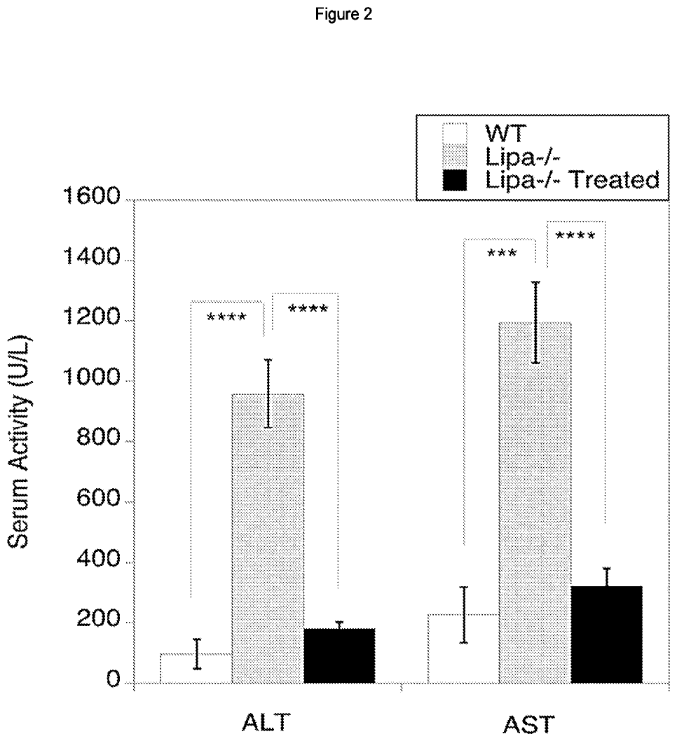

The map for plasmid r(sc) AAVrh74.miniCMV.LIPA is set out in FIG. 2 and the sequence of the entire plasmid is provided in SEQ ID NO: 4. The vector scAAVrh74.miniCMV.LIPA comprises the nucleotide sequence within and inclusive of the ITR's of SEQ ID NO: 4. The rAAV vector comprises the 5′ AAV2 ITR, miniCMV promoter, the coding sequence for the LIPA gene, SV40 late polyA, and 3′ AAV2 ITR. The plasmid set forth in SEQ ID NO: 4 further comprises kanamycin resistance with pUC origin of replication.

Table 2 shows the molecular features of the plasmid (SEQ ID NO: 4), in which range refers to the nucleotides in SEQ ID NO: 4 and [symbol] indicates the kanamycin gene is in the forward orientation.

| Name | Range | Strand | Length |
|---|---|---|---|
| F1 ori | 1158-1465 | [symbol] | 308 |
| LacZ alpha | 1783-1851 | [symbol] | 69 |
| Wt 5′ ITR | 1853-1982 | [symbol] | 130 |
| Mini CMV | 1992-2219 | [symbol] | 228 |
| LIPA transcript | 2254-3453 | [symbol] | 1200 |
| variant 1 | | | |
| SV40 late polyA | 3480-3671 | [symbol] | 192 |
| Wt 3′ ITR (with d TR) | 3679-3906 | [symbol] | 228 |
| ori | 4025-4613 | [symbol] | 589 |
| KanR | 4735-5550 | [symbol] | 816 |

### Example 2

### LIPA Gene Replacement in LIPA-Deficient Mice

The following study tested AAV-based LIPA (human) gene replacement therapy in pure-bred Lipa-deficient (Lipa^{-/-}) mice. Lipa^{-/-}mice, like WD and CESD patients, develop severe liver dysfunction and damage as the result of the massive loading of cholesterol esters and triglycerides into this organ (Du et al. Hum Mol Genet7: 1347-1354, 1998; Du et al. J Lipid Res 42: 489-500, 2001). For example, the mice develop hepatosplenomegaly, elevated serum aspartate aminotransferase (AST), alanine aminotransferase (ALT), and elevated liver and spleen cholesterol and triglycerides. Disease is evident within 3 weeks of birth, and Lipa^{-/-}mice have profound disease by 4 months, showing a 3- to 6-fold increase in the size of the liver and the spleen. Mice succumb to disease several months thereafter, beginning at 6 months of age.

Mice: The lal^{-/-}mice were first generated by Du et al in 1998. The mouse model has been widely used to study the role of Lal in multiple organ systems. The lal^{-/-}mice on a mixed genetic background of 129Sv and CF-1 survive into adulthood, and are fertile, but die at ages of 7 to 8 months. The lal^{-/-}mice show massive accumulation of TGs and CEs in liver, spleen, and small intestine. These mice resemble hepatosplenomegaly, the major clinical manifestation of WD and CESD in human. The lal^{-/-}mice provide a model to study human WD and CESD, but more importantly, serve as a powerful tool to investigate the systemic impacts of lysosomal lipolysis on metabolic and immune homeostasis.

One day-old (P1) Lipa^{-/-}mice were intravenously administered a single injection rscAAVrh74.mCMV.LIPA. 8×10¹³ vg/kg rscAAVrh74.mCMV.LIPA was intravenously administered via the retro-orbital vein. The Lipa^{-/-}mice used were bred on a pure FvB/NJ background, which almost doubled the litter size relative to C57BI/6J-bred mice but did not significantly change disease phenotypes. Without treatment by 4 months of age, livers in Lipa^{-/-}mice have become severely enlarged and damaged.

In the mock-treated Lipa^{-/-}mice at 4 months of age, alanine aminotransferase (ALT) and aspartate aminotransferase (AST) levels mice were elevated 10-fold compared to wild type mice, and these elevations were reduced 90% by AAV treatment (FIG. 2). For both the AST and ALT measures, this was a highly significant change (p<0.0001 for both comparisons). AST and ALT are markers for liver damage, and they are the primary clinical biomarker used to follow LAL-D disease in patients.

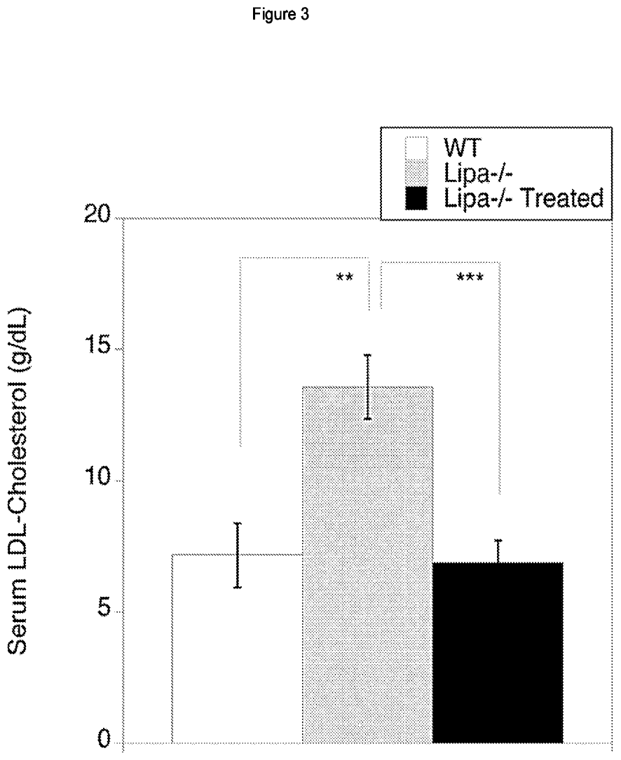

Treatment with rscAAVrh74.mCMV.LIPA also resulted in a complete normalization of serum LDL-cholesterol, which was increased in Lipa^{-/-}mice due to increased export of elevated cholesterol from the liver (FIG. 3). Other serum measures that are impacted by disease, for example reduced serum HDL-cholesterol (which mediates cholesterol influx to the liver) and reduced serum glucose (which likely reflects reduced nutrient uptake into the intestine), both of which are reduced by more than half in 4 mo Lipa^{-/-}mice, were significantly increased towards baseline wild type levels by AAV treatment (not shown). Other measures of abnormal liver function, for example elevated serum total bilirubin and total protein, were also completely corrected by treatment with rscAAVrh74.mCMV.LIPA, though these changes were only statistically significant for total protein (e.g., Tot Bilirubin: WT 0.38±0.02 mg/dL, Lipa^{-/-}0.54±.013 mg/dL, Lipa^{-/-}Treated 0.31±0.04 mg/dL; Tot protein: WT 5.55±0.07 g/dL, Lipa^{-/-}6.82±0.10 g/dL (p<0.00001 vs. WT), Lipa^{-/-}Treated 5.78±0.18 (p<0.001 vs. untreated Lipa^{-/-}).

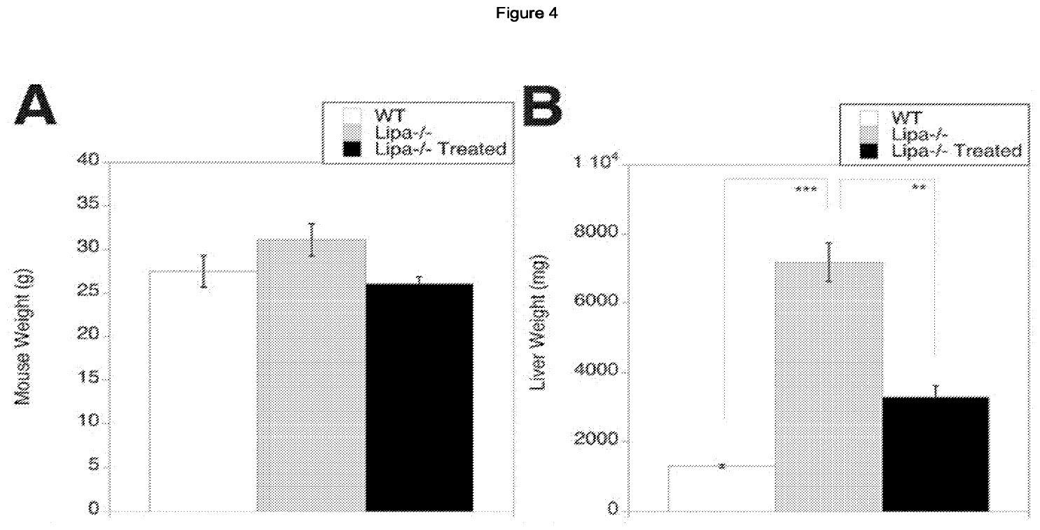

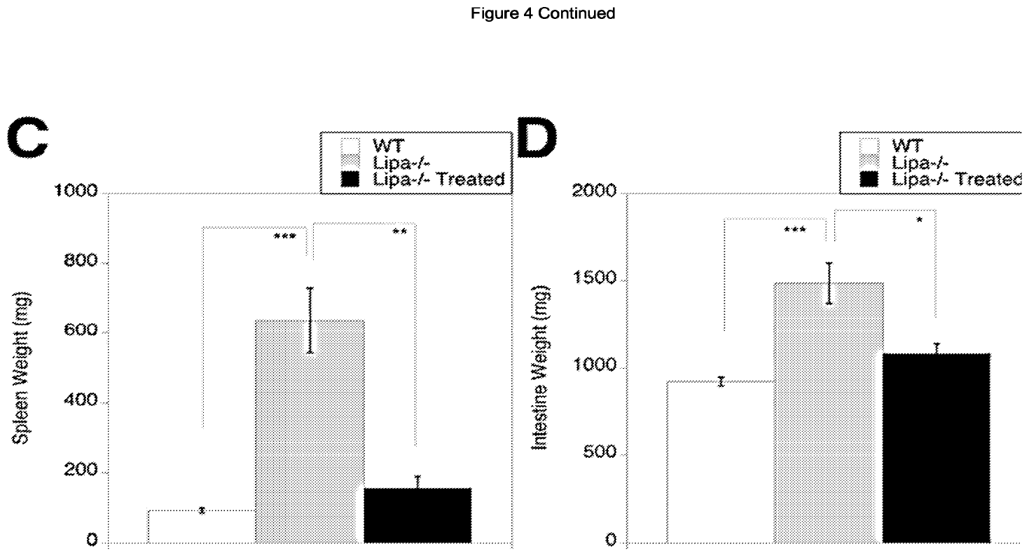

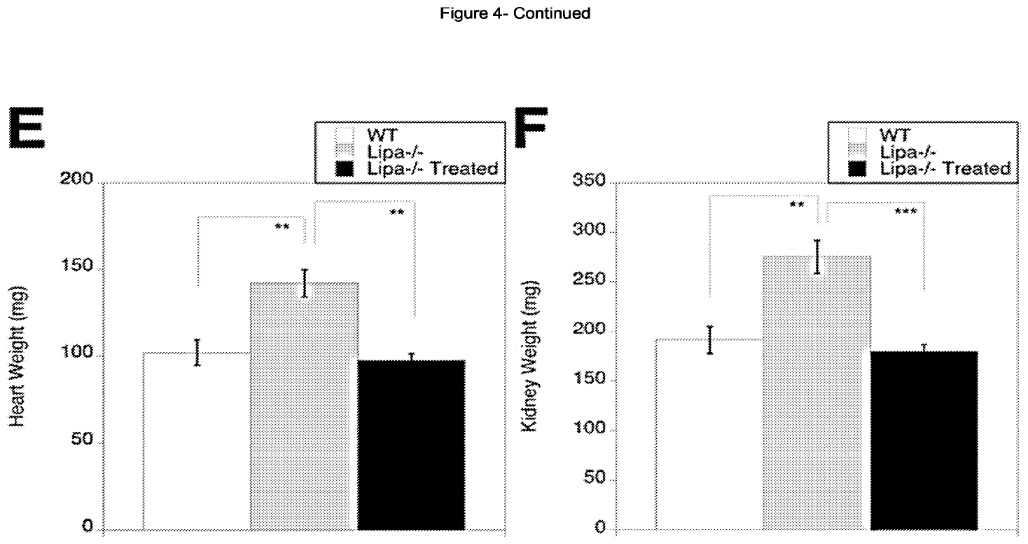

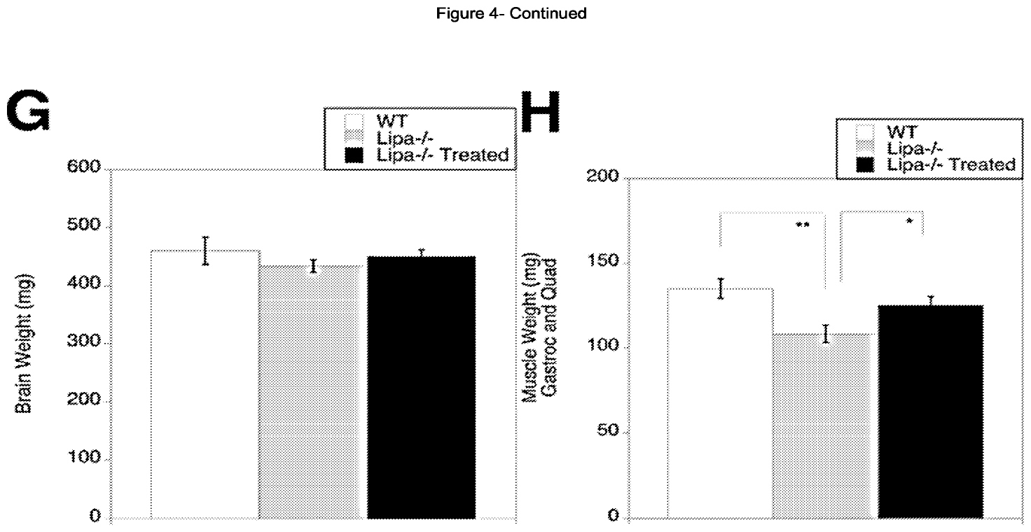

By 4 months of age, Lipa^{-/-}mice are easily recognizable by sight, as they have enlarged and distended abdomens due to the dramatic increase in the size of the liver and spleen, a symptom of the human disease as well (Burton et al. J Pediatr Gastroenterol Nutr 61: 619-625, 2015). By the 6-month endpoint, both the spleen and the liver had increased more than 5-fold in total size (FIG. 4). This is largely due to the profound loading of triglycerides into these organs, but other organs were also affected; intestine, heart, and kidney, for example, show increased size due to fat loading (FIG. 4). Brain size, by contrast, is not increased, while muscle size decreased, most likely due to impaired nutrient absorption (FIG. 4). Treatment with the 8×10¹³ vg/kg dose of rscAAVrh74.mCVM.LIPA reduced the increase in organ size by at least 70% at 6 months of age in all tested organs, with all such organs showing a significant improvement toward their baseline wild type measures (FIG. 4).

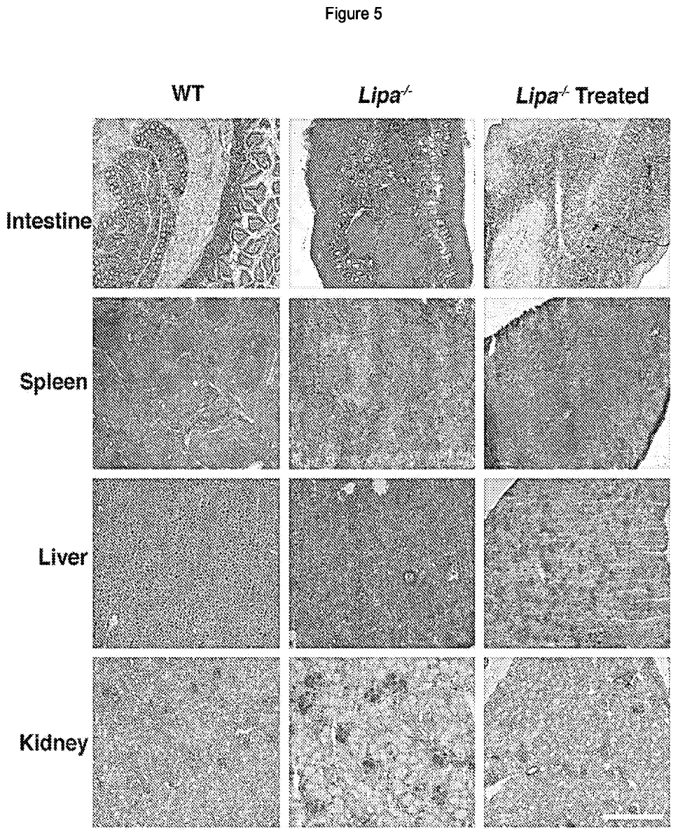

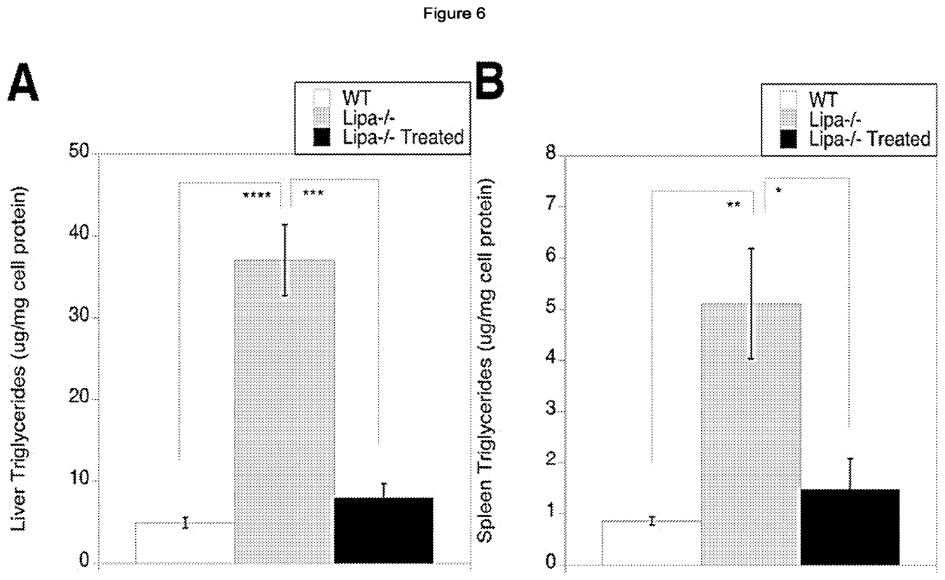

These improvements in organ size were matched by significant reductions in staining using Oil Red O (with hematoxylin counterstain in blue, FIG. 5). Oil Red O staining, to identify lipid burden in intestine, spleen, liver, and kidney, showed very significant decreases in staining in AAV-treated Lipa^{-/-}mice compared to mock-treated Lipa^{-/-}controls at 6 months of age. Such decreased staining was highly suggestive of reduced fat content. This was confirmed with biochemical measures of triglyceride content in liver and spleen. Here, the more than 5-fold increase in triglyceride content in untreated Lipa^{-/-}mice was significantly reduced by AAV treatment, approaching baseline wild type levels in both instances (FIG. 6). AAV treatment also led to improvements in the overall activity levels of mice on open field tests, where mice showed as much or more ambulation activity than wild type mice. For example, at 6 months, average total ambulatory events per five-minute interval were decreased by 35% in mock-treated Lipa^{-/-}mice compared to wild type FVB/NJ controls but this measure was increased to 1.35 times wild type activity in AAV-treated Lipa^{-/-}mice (WT: 4477±381 events/5 min, Lipa^{-/-}:2925±771 events/5 min, Lipa^{-/-}Treated: 6069±72 events/5 min, p<0.05 for treated vs. untreated Lipa^{-/-}comparison).

Surprisingly, while it was expected that the liver would be easily transduced with AAV in Lipa^{-/-}mice, the liver was only transduced with 1.7±0.4 vg/nucleus at the 8×10¹³ vg/kg IV dose (n=4), a mere 2-fold elevation in transduction relative to skeletal muscle in the same mice (0.9±0.1 vg/nucleus, n=4). While this level of transduction suggests saturation of cells in the liver (>1 vg/nucleus), it is not uncommon with other AAV vectors at this dose to generate 10-100 vg/nucleus [22]. This lower level of transduction is likely due to the fact that hepatocytes in the liver divide in the first month of life in mice as the liver grows, making it impossible to transduce such cells with treatment at postnatal day 2, a time prior to the genesis of such liver cells.

### Example 3

### Gene Therapy of Young Adult Mice

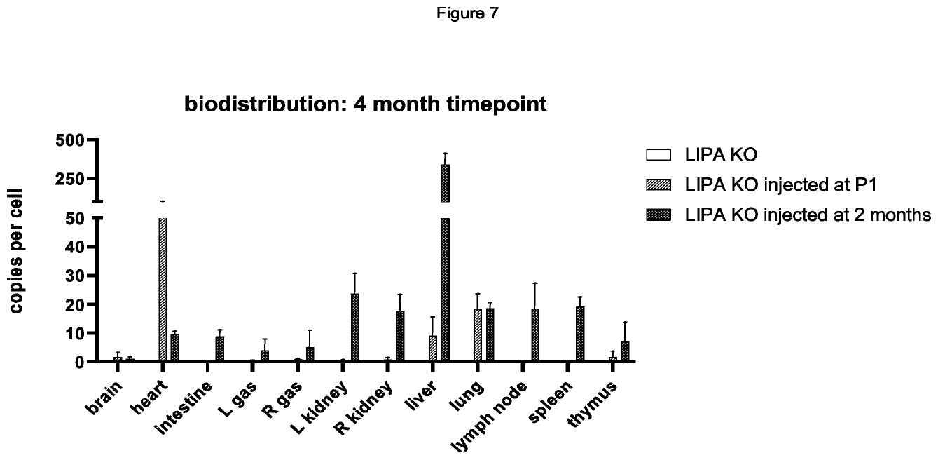

Because hepatocytes divide in the liver as it grows until about one month of age, treatment of young adult (2-month-old) mice was investigated. The 2-month Lipa^{-/-}mice were intravenously administered a single injection rscAAVrh74.mCMV.LIPA. 8×10¹³ vg/kg rscAAVrh74.mCMV.LIPA via the retro-orbital vein. Treatment of adult Lipa^{-/-}mice with rscAAVrh74.mCMV.LIPA led to a logarithmic increase in transduction of viral genomes into the liver, increasing from 2 to 50 vg/nucleus (FIG. 7).

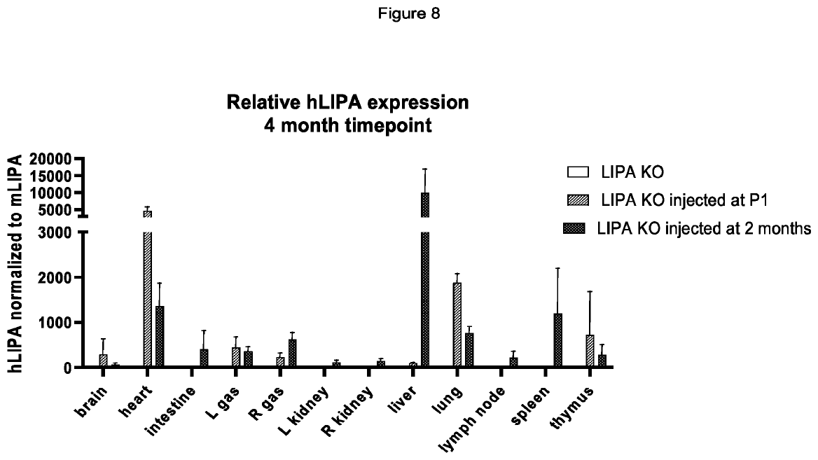

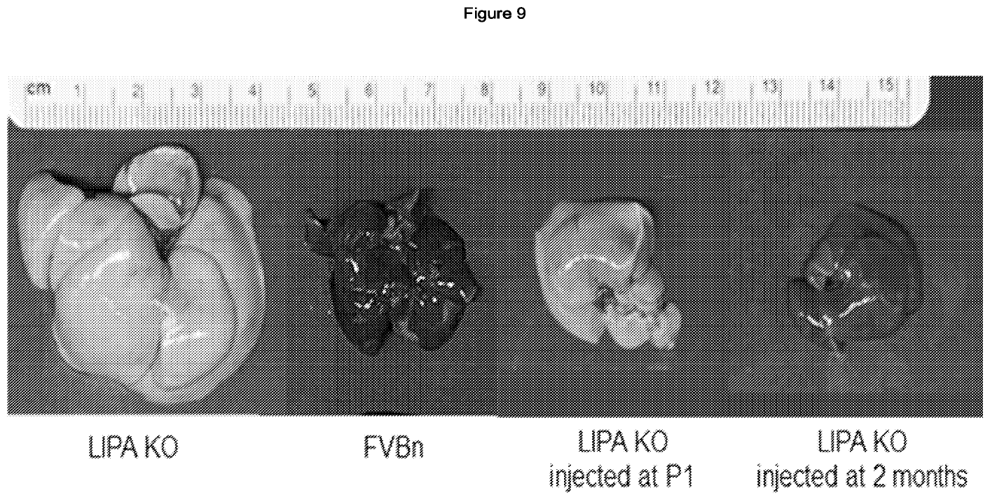

This level of transduction led to an increased human LIPA transgene expression exceeded normal mouse LIPA gene expression found in wild type tissues, as measured by qRT-PCR (FIG. 8). The overall appearance of the liver from a 4 mo Lipa^{-/-}mice treated at 2 months of age was no different than wild type (FVBn), showing no fat whatsoever, while a 4 mo Lipa^{-/-}from a mouse treated at P1 still contained some fat, though not nearly as much as an untreated Lipa^{-/-}mouse, which was also greatly increased in size (FIG. 9).

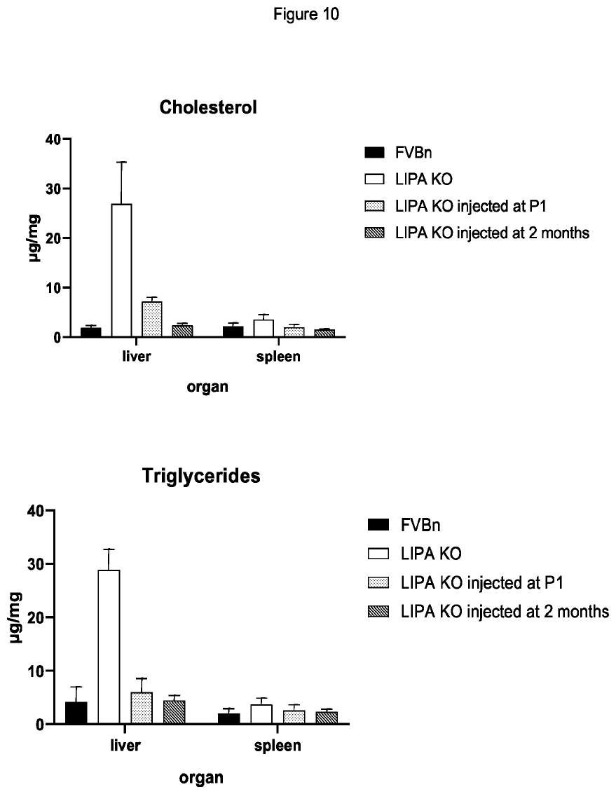

The reduced fat content seen visually was confirmed by the measured reduction in cholesterol and triglycerides in treated Lipa^{-/-}(LIPA KO) mice in both liver and spleen, which in the 2 mo condition were not significantly different from wild type (FIG. 10).

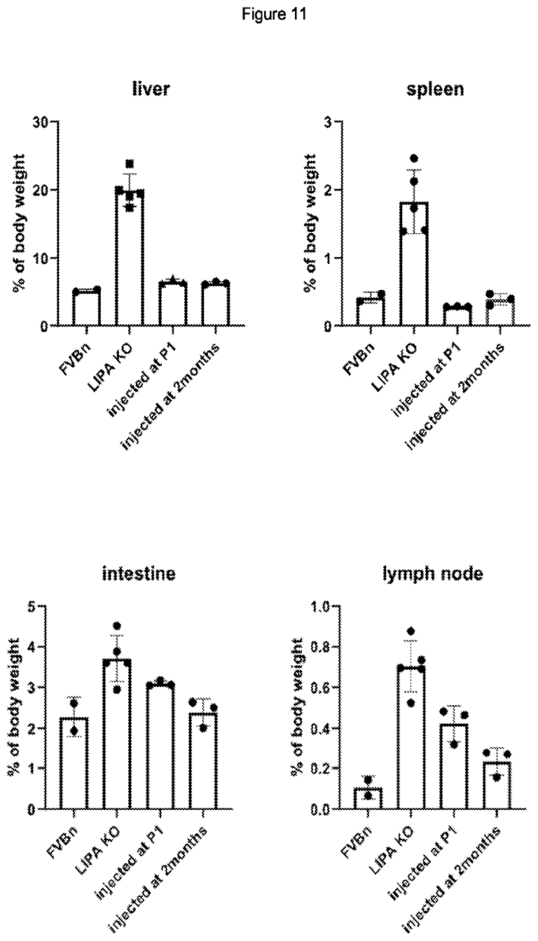

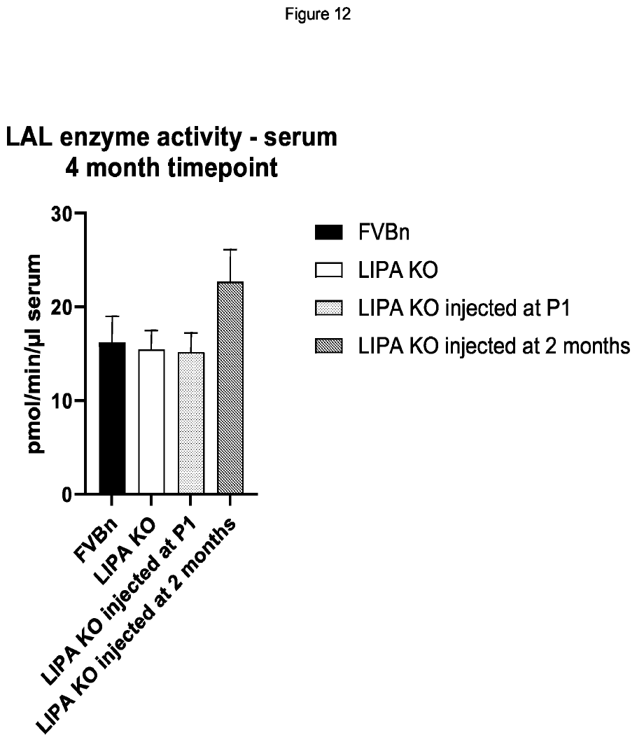

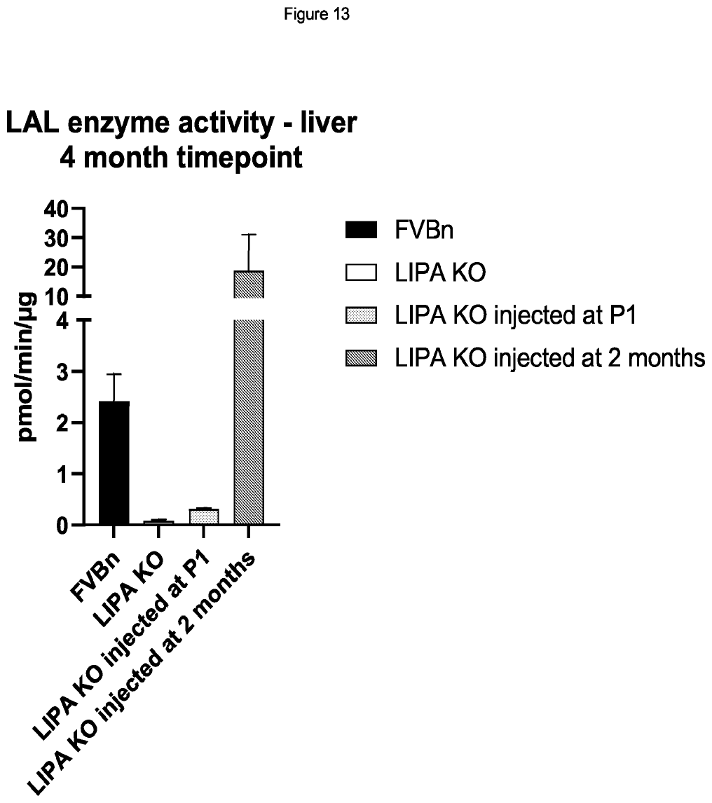

The weights of liver, spleen and intestine were all reduced to near normal levels with treatment at 2 months, despite the fact that these organs had already doubled in size due to fat burden by this point in untreated Lipa^{-/-}mice (FIG. 11). Thus, rscAAVrh74.mCMV.LIPA can both prevent and reverse disease symptoms in a LAL-D model. Blood cells in lymph nodes showed greater prevention of fat burden with treatment at 2 mo compared to 2 days (FIG. 11). As mouse blood cells turn over in the course of months, this suggests a trans effect of rscAAVrh74.mCMV.LIPA treatment in adult mice, as blood cells would have lost AAV expression due to turnover by 4 months of age. This finding correlated with the fact that LIPA enzyme activity was actually elevated in the serum of Lipa^{-/-}mice treated with rscAAVrh74.mCMV.LIPA as adults (FIG. 12), along with its elevation in liver tissue (FIG. 13).

This data suggest that LIPA enzyme is released from the liver and is expressed systemically in the blood as the result of gene therapy treatment in the adult animal, providing therapeutic protein in trans to tissues where cells turn over constantly, such as the blood. This allows rscAAVrh74.mCMV.LIPA gene therapy to also provide enzyme replacement therapy to transduced cells and tissues when the proper dose and timing of therapy is given, but also to cells not transduced by the gene therapy. Such a therapy would allow for constant prevention of disease throughout the whole body on an ongoing basis during the patient's life.

### Example 3

### rscAAVrh74.miniCMV.LIPA Treatment Can Inhibit and Reverse Hepatosplenomegaly and Elevated Serum Liver Enzymes in Lipa^{-/-}Mice

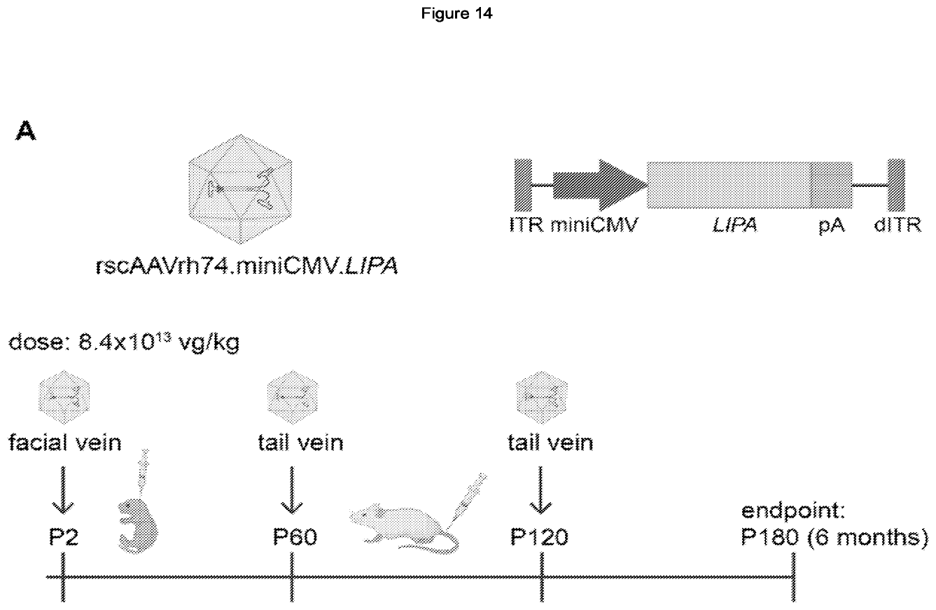

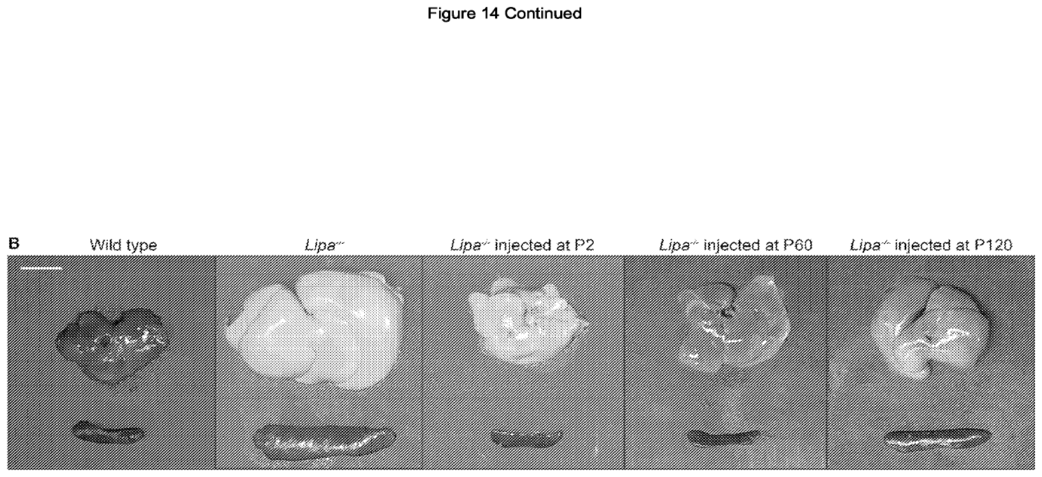

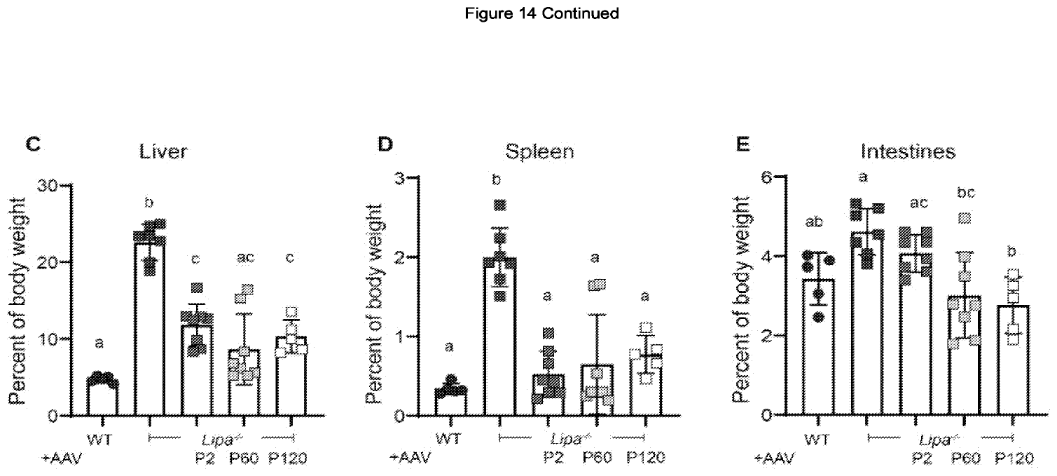

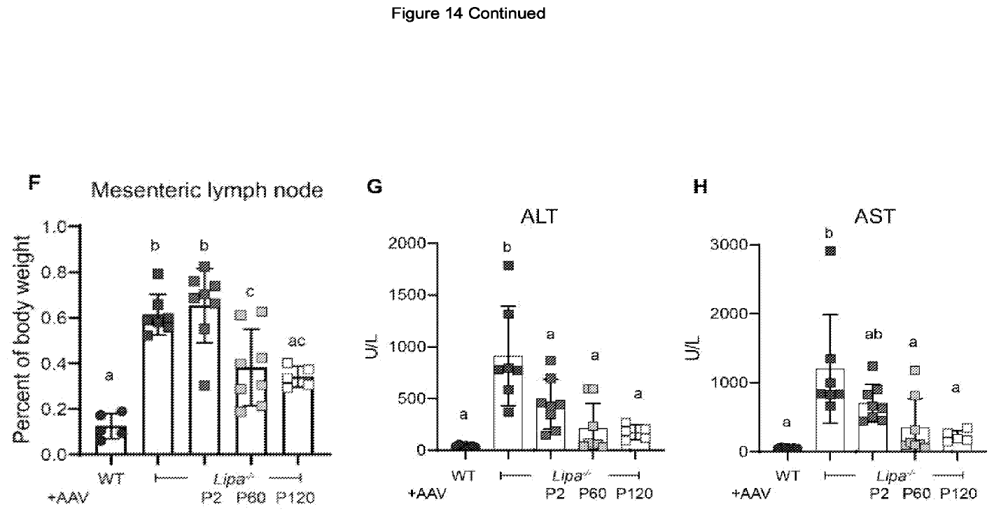

As an extension to the experiments described in Example 2, rscAAVrh74.miniCMV.LIPA was administered at various ages. These experiments tested an AAV dose that, when taking into account differences in titering methods, was equivalent to the highest doses currently being used in gene therapy clinical trials (36, 37). A dose of 8.4×10¹³ vector genomes (vg) per kilogram (vg/kg) of rscAAVrh74.miniCMV.LIPA AAV gene therapy was intravenously injected via the facial vein at early (P2, postnatal day 2) or via tail vein at middle (P60, postnatal day 60) or advanced (P120, postnatal day 120) disease stages in Lipa^{-/-}mice. Mice were followed to endpoints of P60 (2 months of age), P120 (4 months of age) and P180 (6 months of age) (FIG. 14A). At these time points, mice were necropsied, organs (liver, spleen, kidneys, intestine, mesenteric lymph node, heart, lung, thymus, brain) and muscles (left and right gastrocnemius and quadriceps) were harvested for biodistribution and gene expression. Harvested non-muscle organs were weighed and then immersed in OCT before being frozen in dry ice-cooled isopentane. Muscles were weighed and then snap-frozen in liquid nitrogen-cooled isopentane.

Hepatosplenomegaly, or the enlargement of the liver and spleen, and yellowing of organs due to increased fat deposition are both defining features of LAL-D and of disease in Lipa^{-/-}mice. Both phenotypes were present and progressed with age in Lipa^{-/-}mice (FIG. 14B-D). Liver weight increased over time to comprise as much as 25% of total body weight by 6 months of age, in contrast to being only 5% of body weight at all 3 ages in wild type (WT) mice (FIG. 14C). Similarly, spleen weight increased, on average, to 2% of total body weight at 6 months of age in Lipa^{-/-}mice compared to 0.3% in WT (FIG. 14D,). Treatment of Lipa^{-/-}mice at P2, P60, and P120 resulted in reduced liver weight and normalized liver color, but early treatment (P2) was far less effective than treatment at P60 or P120. P2 injection reduced liver size to wild-type levels at 2 and 4 months, suggesting disease inhibition, but only partially reduced liver weight, by about 50%, at 6 months. By contrast, P60 and P120 injections were more effective despite already present evidence of hepatosplenomegaly at these ages, reducing liver weight to 8% and 10% of total body weight, respectively, at 6 months, suggesting disease reversal. P2 injection was more effective at reducing spleen size than it was for liver at the 6-month endpoint (FIG. 14D), and treatment at all 3 time points reduced spleen weight to near wild type levels. Intestines and mesenteric lymph nodes also showed increased weight in Lipa^{-/-}mice (FIG. 14E, F). Weight in intestine was increased to 4.5% of total body weight at 6 months of age compared to 3% in WT (FIG. 14E), while weight of mesenteric lymph node was increased to 0.6% of body weight compared to 0.1% in WT (FIG. 14F). Here again, AAV treatment reduced intestine and mesenteric lymph node weight in a manner similar to responses seen with liver, with P2 injection showing improvement to near wild type levels at 2 or 4 months that was lost by 6 months, while injection at P60 or P120 showed reductions in weight at the 6-month endpoint. Unlike liver, spleen, and intestine, there were no data that showed a complete normalization of lymph node weight at any time point with any of the treatment times tested. Instead, at best, only a 50% average reduction in weight was achieved by 6 months (FIG. 14F).

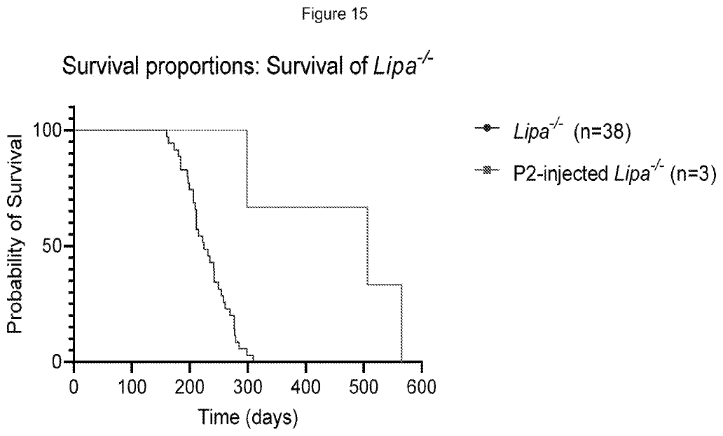

Serum ALT and AST activity were also measured, and these enzyme when elevated indicate liver damage (FIG. 14G, H). Both serum ALT and AST levels were elevated 20-fold in Lipa^{-/-}mice compared to WT by 6 months. Here, treatment at all time points resulted in decreased serum ALT/AST levels, with a more pronounced effect with injection at P60 and P120 than at P2. As with liver weights, injection at P2 reduced serum ALT and AST levels at the 2- and 4-month time points to near wild-type levels, but showed only about a 50% reduction at 6 months . Injection of this dose of rscAAVrh74.miniCMV.LIPA in WT mice did not significantly elevate serum transaminase levels at any of the time points tested. Despite the increased weight and serum liver enzyme levels at 6 months for P2 injection, the several P2-treated Lipa^{-/-}mice that lived beyond the 6-month endpoint showed a significant increase in overall lifespan relative to untreated Lipa^{-/-}controls (FIG. 15). Untreated Lipa^{-/-}mice showed a median survival of 224 days, whereas the P2-treated Lipa^{-/-}mice had a median survival of 506 days, more than double the median life span of untreated Lipa^{-/-}mice.

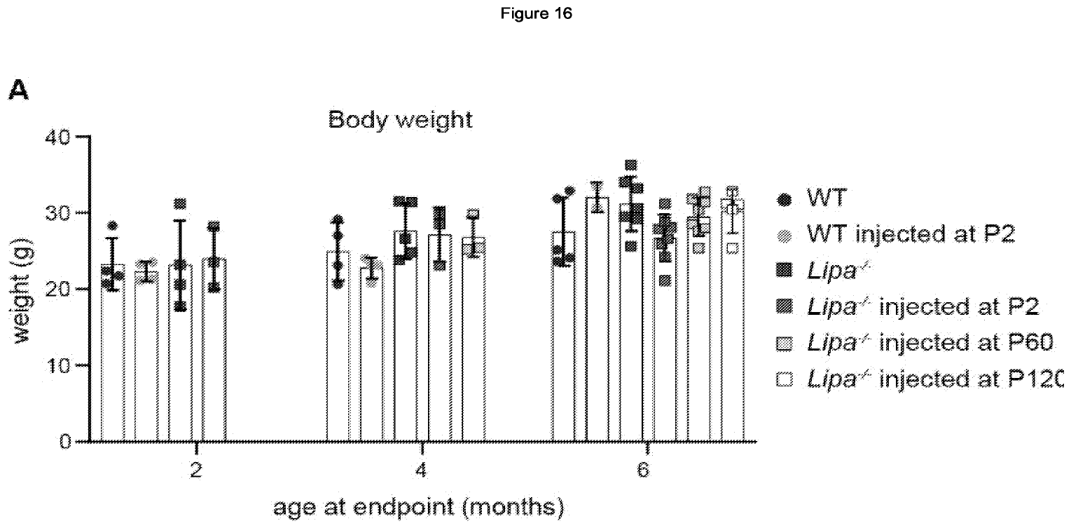

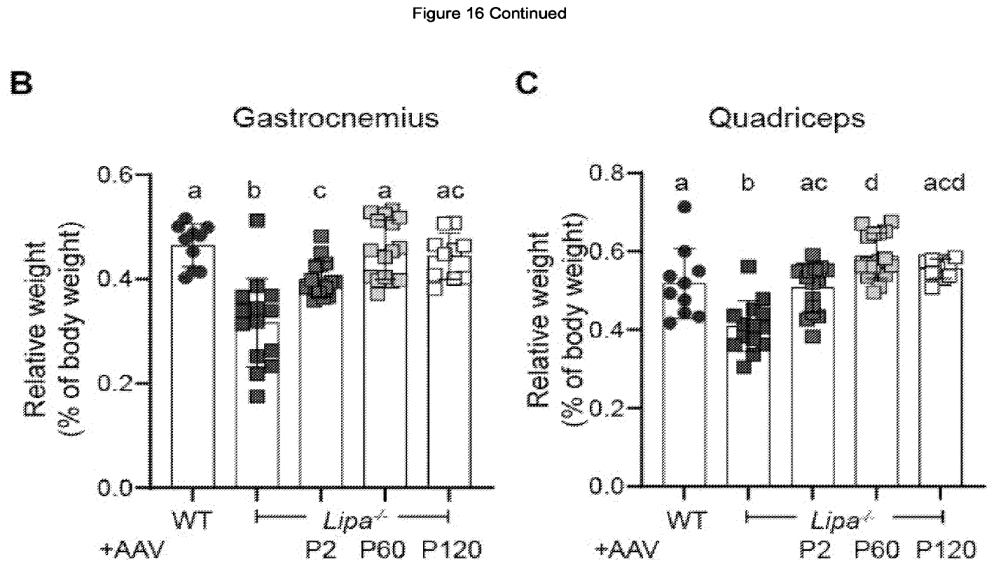

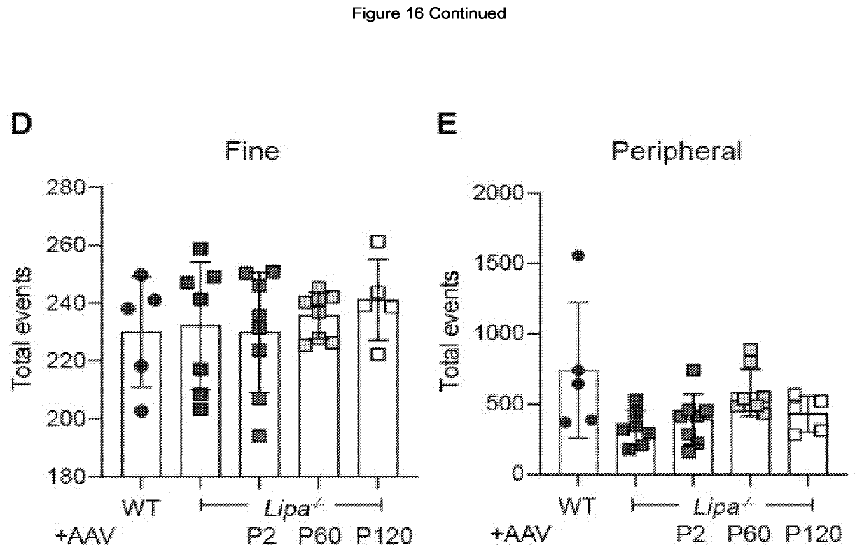

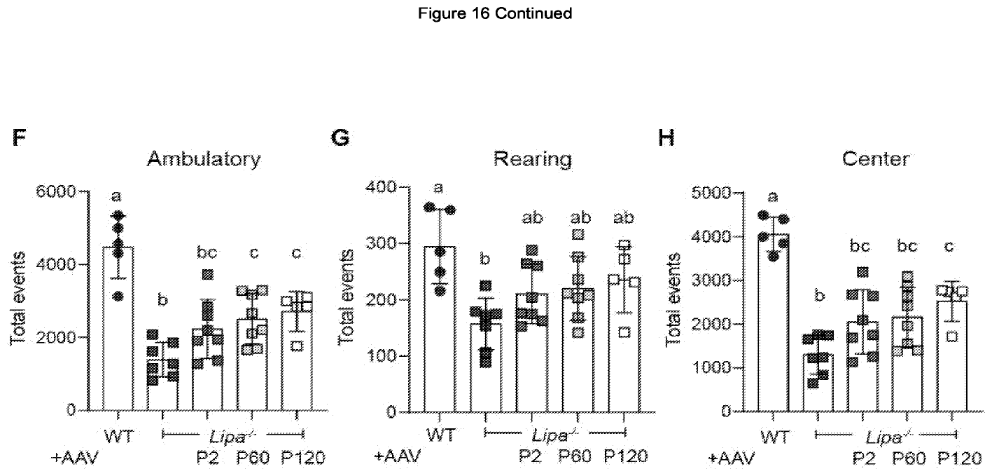

The body weight of Lipa^{-/-}mice did not differ from that of WT mice at any study time point (FIG. 16A). Additionally, a significant reduction in muscle mass was observed in the Lipa^{-/-}mice, about 25% (FIG. 15B, C). Due to the enlarged liver, Lipa^{-/-}mice presented with a distended abdomen. Open field studies which determined fine, ambulatory, center, peripheral, and rearing movement events were performed as previously described (49) were carried out to determine if mobility was affected (FIG. 16D-H). Fine movement (such as sniffing and grooming) and peripheral movement (movement at the periphery of the open field area) were not affected in Lipa^{-/-}mice relative to WT, but center-cage based ambulation and rearing were significantly decreased. All such measures were improved with treatment at all time points. Such muscle atrophy may also contribute to ambulation differences in additional to abdominal distension in Lipa^{-/-}mice, and these muscle weights were increased back to WT levels with P60 and P120 injection, while they only were marginally increased for P2 (Supplemental FIG. 16B, C).

### Example 4

### AAV Transduction in Organs Results in LIPA Expression and Restored Lysosomal Acid Lipase Enzyme Activity

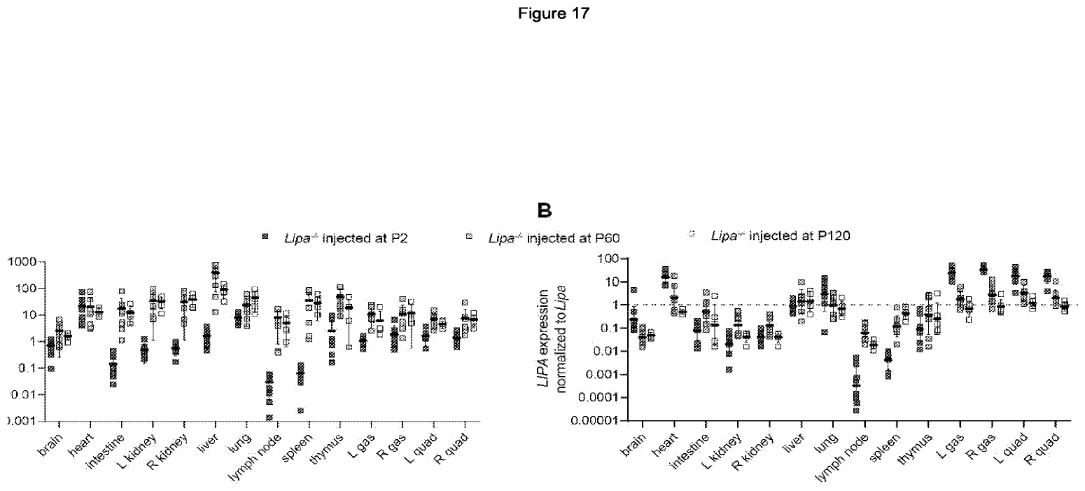

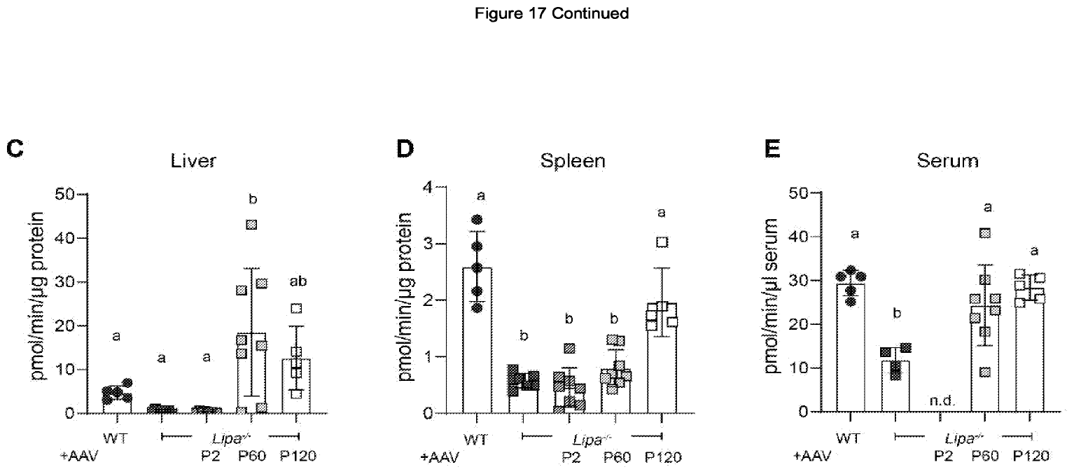

Biodistribution of AAV vgs was assessed in various organs and tissues from all injection time points using quantitative real-time PCR (FIG. 17A) as described in (50). At the 6-month endpoint, there was a difference in organ biodistribution between mice injected at P2 versus mice injected at P60 or P120; injections at P2 showed greater AAV transduction in the heart and lungs (21.82±8.41 vg/nucleus, and 7.87±1.16 vg/nucleus, respectively; Table 3 while injection at P60 or P120 showed greater transduction in the liver (406.13±117.59 vg/nucleus and 90.69±21.48 vg/nucleus, respectively), with higher levels (20-50 vg/nucleus) also in kidney, lung, spleen, and thymus (FIG. 17A, Table 4). Comparison of kidneys and muscles from the left and right side of the body plan suggested even AAV distribution. With the injections at P2, transduction levels were relatively stable over the course of the study in most organs, though there was a sharp decline in transduction between 4 and 6 months in the heart and liver (Tables 3 and 4, below). With injections at P60, transduction levels were also stable when measured at 2- and 4-months post-injection.

TABLE 3
Biodistribution of AAV at 6-month endpoint. Values
are represented as mean vg/nucleus ± SD.

| | Injected at | Injected at | Injected at |
|---|---|---|---|
| | P2 (n = 8) | P60 (n = 8) | P120 (n = 5) |
| Brain | 1.20 ± 0.14 | 2.55 ± 0.81 | 1.64 ± 0.21 |
| Heart | 21.82 ± 8.41 | 20.52 ± 8.79 | 12.9412.11 |
| Intestines | 0.14 ± 0.05 | 17.93 ± 9.08 | 12.58 ± 3.73 |
| Liver | 1.62 ± 0.39 | 406.13 ± 117.59 | 90.69 ± 21.48 |
| Lung | 7.87 ± 1.16 | 23.91 ± 7.15 | 45.47 ± 14.73 |
| Lymph node | 0.03 ± 0.01 | 8.14 ± 2.59 | 4.99 ± 1.94 |
| L kidney | 0.48 ± 0.12 | 34.95 ± 11.97 | 33.00 ± 6.93 |
| R kidney | 0.55 ± 0.09 | 30.66 ± 10.44 | 38.83 ± 8.58 |
| Spleen | 0.07 ± 0.02 | 35.66 ± 13.19 | 28.31 ± 9.92 |
| Thymus | 2.52 ± 1.28 | 49.44 ± 14.81 | 19.25 ± 8.36 |
| L gastrocnemius | 1.10 ± 0.15 | 10.75 ± 2.66 | 6.19 ± 3.48 |
| R gastrocnemius | 1.86 ± 0.73 | 10.79 ± 4.61 | 11.89 ± 5.07 |
| L quadricep | 1.65 ± 0.33 | 7.11 ± 1.48 | 4.55 ± 0.65 |
| R quadricep | 1.38 ± 0.25 | 7.50 ± 3.29 | 6.81 ± 1.53 |

TABLE 4
Biodistribution of AAV at the 2- and 4- month endpoints.
Values are represented as mean vg/nucleus ± SD

| | Injected | Injected | Injected |
|---|---|---|---|
| | at P2, 2 | at P2, 4 | at P60, 4 |
| | month | month | month |
| | endpoint | endpoint | endpoint |
| | (n = 3) | (n = 3) | (n = 3) |
| Brain | 0.28 ± 0.06 | 1.70 ± 1.09 | 1.13 ± 0.48 |
| Heart | 47.27 ± 4.76 | 74.34 ± 18.01 | 0.97 ± 0.61 |
| Intestines | 0.05 ± 0.01 | 0.11 ± 0.02 | 8.97 ± 1.33 |
| Liver | 10.73 ± 7.29 | 9.21 ± 4.09 | 340.12 ± 37.33 |
| Lung | 6.85 ± 1.01 | 18.42 ± 3.32 | 18.61 ± 1.25 |
| Lymph node | 0.46 ± 0.45 | 0.02 ± 0.00 | 18.55 ± 5.58 |
| L kidney | 0.45 ± 0.09 | 0.66 ± 0.18 | 23.74 ± 4.53 |
| R kidney | 0.51 ± 0.05 | 0.84 ± 0.45 | 17.78 ± 3.59 |
| Spleen | 0.23 ± 0.07 | 0.08 ± 0.01 | 19.26 ± 1.79 |
| Thymus | 1.15 ± 0.95 | 1.63 ± 1.44 | 7.09 ± 4.36 |
| L gastrocnemius | 2.92 ± 0.60 | 0.54 ± 0.06 | 4.09 ± 2.35 |
| R gastrocnemius | 2.23 ± 0.95 | 0.95 ± 0.10 | 5.08 ± 3.09 |
| L quadricep | 0.78 ± 0.15 | 0.74 ± 0.29 | 0.91 ± 0.13 |
| R quadricep | 0.67 ± 0.10 | 0.68 ± 0.07 | 0.98 ± 0.14 |

Next, quantitative reverse transcription (RT)-PCR was used to measure mRNA levels of the human LIPA transgene in treated mice compared to normal expression of endogenous mouse Lipa gene in wild-type tissues. At the 6-month study endpoint, there was 3-fold and 15-fold LIPA expression in the lung and heart, respectively with injection at P2 (FIG. 17B), consistent with the finding of 7 and 20 vg/nucleus of AAV vgs in these organs at this time point. P2 injection achieved a near normal level of gene expression in liver, with human LIPA/mouse WT Lipa equal to 0.8. Interestingly there was only a 1.5-fold increase in liver in the same ratio for P60 or P120 injection at 6 months, despite the fact that there was, on average, 90 or 400 vgs/nucleus in liver at this time point, respectively, suggesting feedback inhibition on AAV-mediated LIPA transgene expression. Similarly, AAV transduction in the kidneys and lymph node did not result in increased expression LIPA/Lipa ratios despite there being 5-30 vg/nucleus in those tissues at the P60 and P120 time points. Also of interest was the fact that P2-injected skeletal muscles (gastrocnemius and quadriceps femoris) showed a 20-fold LIPA/Lipa ratio at the 6-month time point for P2, while P60 and P120 injected muscles showed a lower ratio despite having higher AAV vgs. rscAAVrh74.miniCMV.LIPA treatment at any time point did not alter endogenous mouse Lipa expression (not shown), but tissues in Lipa^{-/-}mice did show some reductions in endogenous Lipa gene expression relative to wild type (not shown).

Lysosomal acid lipase (LAL) enzyme activity in liver, spleen, and serum from treated and control mice was also measured (FIG. 17C-E). Overall LAL enzyme activity was reduced by 90% in Lipa^{-/-}liver relative to WT (FIG. 17C). At the 6-month endpoint, liver enzyme activity did not significantly differ between the untreated Lipa^{-/-}mice and those treated at P2 at all examined time points (FIG. 17C). When injected at P60 and P120, however, AAV treatment led to a significant increase in enzyme activity that exceeded WT activity by 4-fold and 2.5-fold, respectively. In spleen, overall LAL activity was reduced about 80% relative to WT at 6 months. Similar to liver, P2-injected spleens showed no improvement in activity at 6 months. Unlike liver, however, the P60 treatment time point in spleen also showed no improvement, while P120 did show an increase, though one that did not reach WT levels (FIG. 17D).

Serum LAL enzyme activity was also assayed to determine whether exogenous LAL was being secreted from cells as a result of treatment in a manner that might be utilized in trans by other tissues. Frozen liver and spleen samples were homogenized in LAL tissue extraction buffer (0.1 M sodium phosphate pH 6.8, 1 mM EDTA, 0.02% sodium azide, 10 mM DTT, 0.5% NP-40). Protein concentrations were determined with the bicinchoninic acid assay (Pierce), using BSA as the standard. LAL activity was determined using 4-methylumbelliferyl palmitate (4-MUP; Gold Biotechnology) as the substrate, as previously described (51, 52). Briefly, 1 μg of protein was added to 0.345 mM substrate solution (0.345 mM 4-MUP, 90.9 mM sodium acetate, pH 4.0, 1% (v/v) Triton X-100 and 0.0325% (w/v) cardiolipin), and enzymatic reactions were performed in triplicate in the presence or absence of the LAL inhibitor Lalistat2 (Sigma Aldrich). Reactions were incubated at 37° C. for 3 hours in the dark. Reactions were terminated by adding 200 μl of 150 mM EDTA, pH 11.5. A standard curve was prepared ranging from 0-33.3 μM 4-methylumbelliferone (4-MU; Gold Biotechnology). Fluorescence was measured on a SpectraMax M2 plate reader (Molecular Devices) using a 355-nm excitation filter and a 460-nm emission filter. LAL activity (pmol/min/μg) was calculated by subtracting the enzymatic activity of the inhibited reaction from that of the-uninhibited reaction.

Serum enzyme activity was only performed on mice treated at P60 and P120, as there was not enough serum remaining after blood chemistry analysis from P2-treated mice. Overall LAL activity in untreated LIPA^{-/-}mice was about one third that of the WT (11.80±2.89 vs 29.43±2.91 pmol/min/μl serum). With treatment at P60 and P120, serum LAL enzyme activity was restored to WT levels (FIG. 17E).

### Example 5

### Triglyceride and Cholesterol Levels are Reduced in rscAAVrh74.miniCMV.LIPA-treated Lipa^{-/-}Mice

Total lipids were extracted from snap-frozen tissues using the Lipid Extraction Kit (Chloroform Free) (abcam) per manufacturer's protocol. Triglycerides were measured using the Infinity Triglycerides Reagent (Thermo Fisher Scientific), and total cholesterol was measured using the Infinity Cholesterol Reagent (Thermo Fisher Scientific). Lipid concentrations were determined against a standard curve of triglycerides or cholesterol standards (Pointe Scientific). Measurements were performed in triplicate, and absorbance values at 500 nm were measured on a Synergy 2 plate reader (BioTek Instruments).

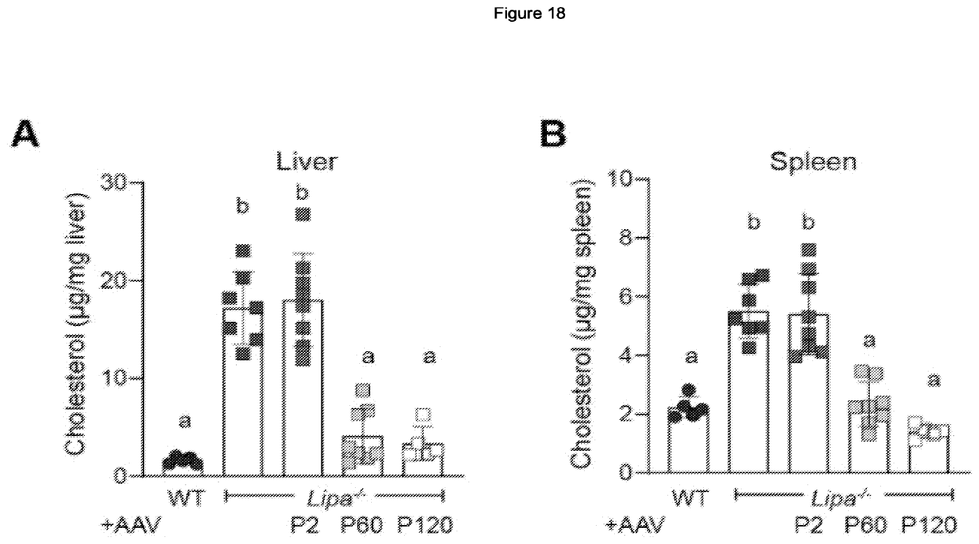

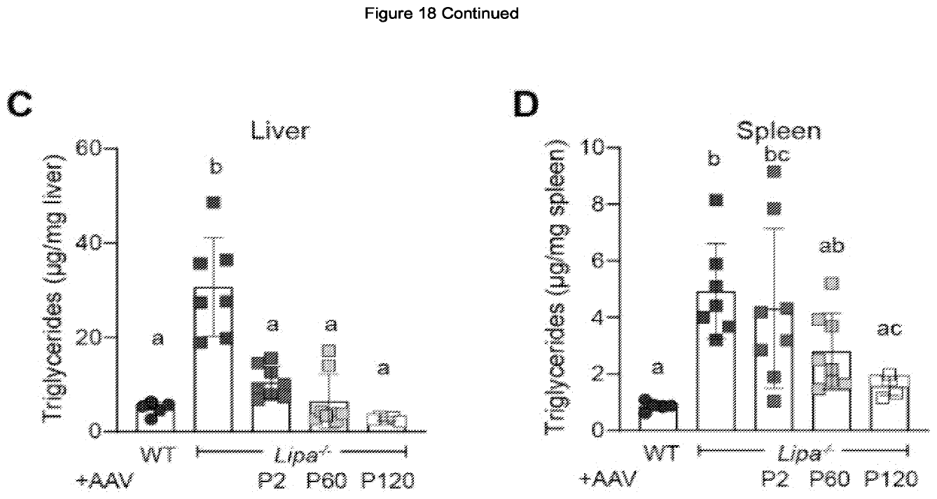

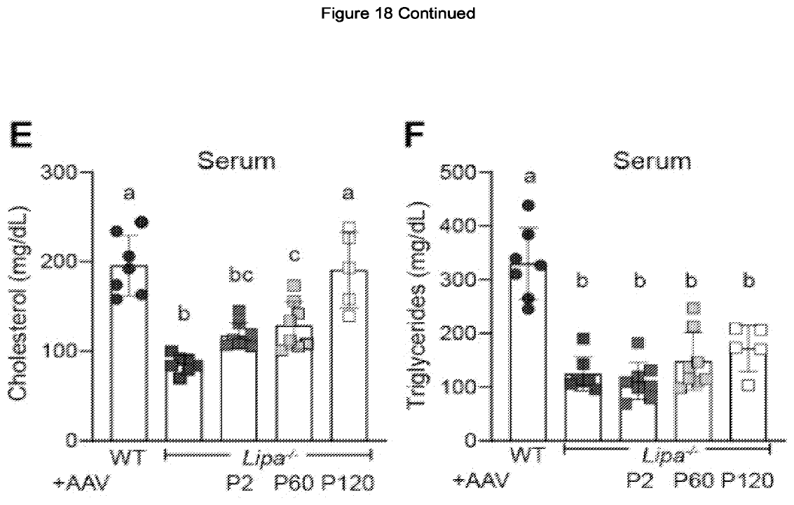

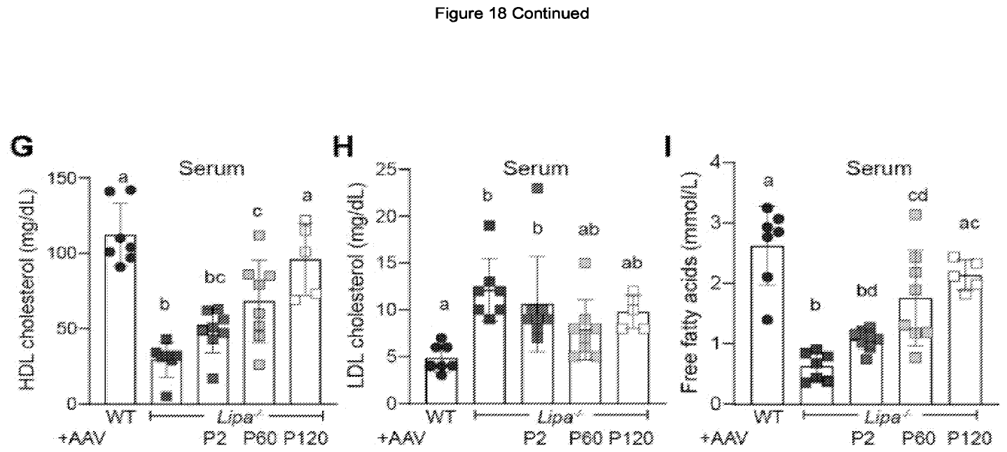

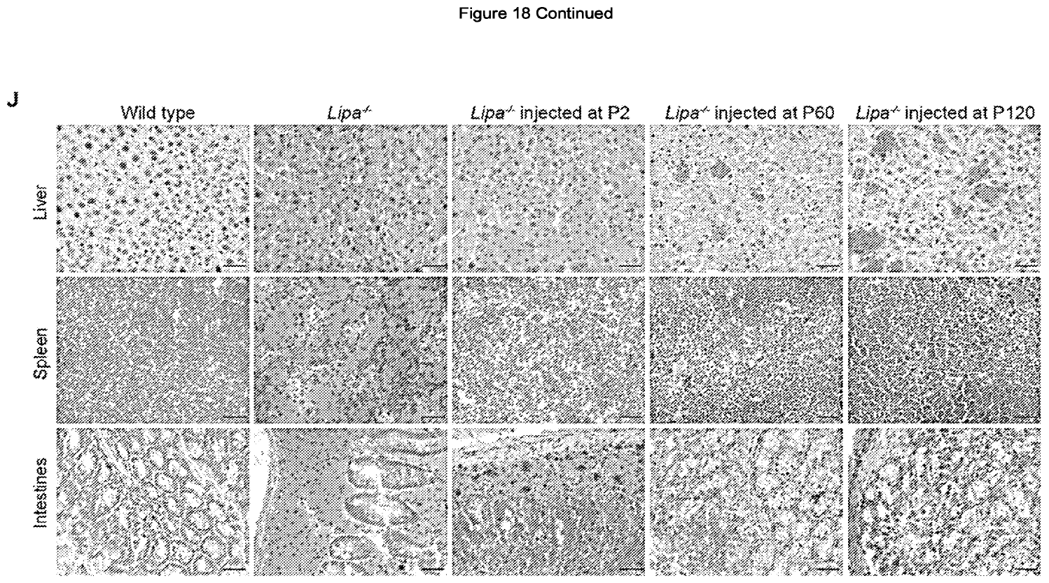

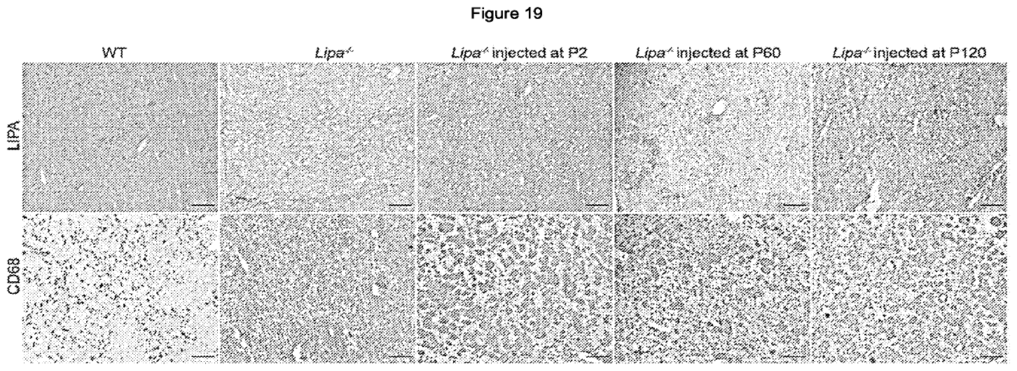

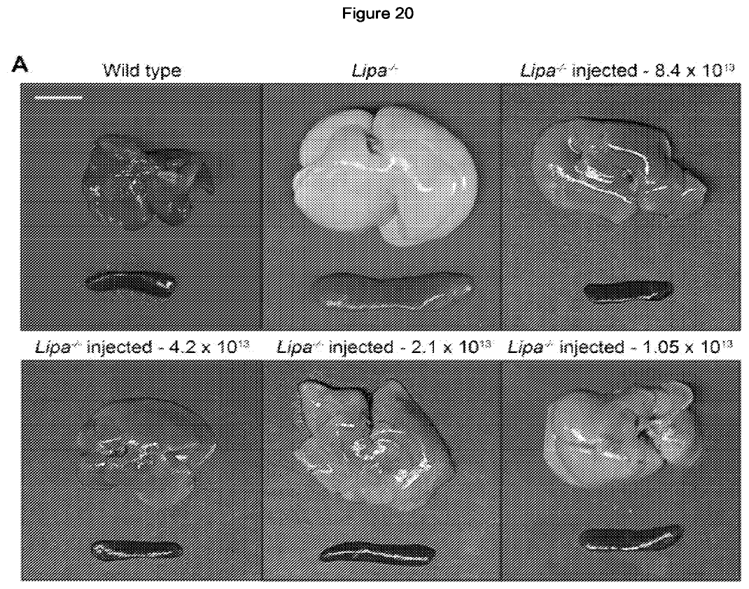

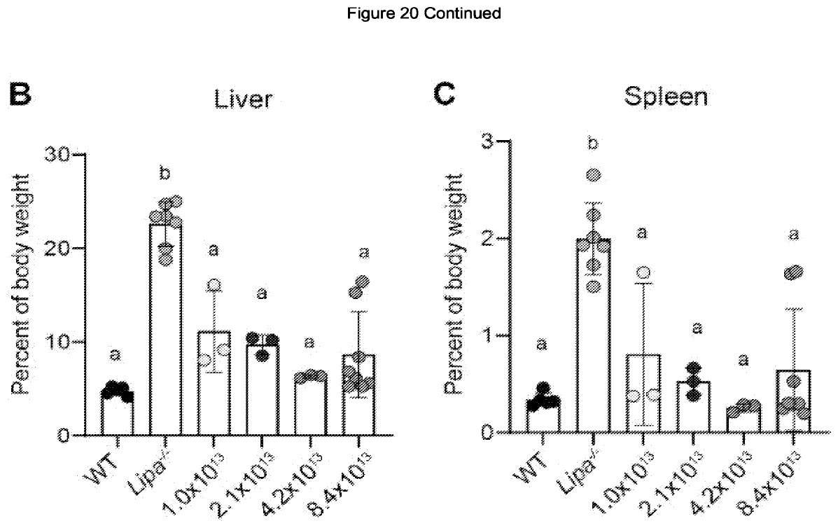

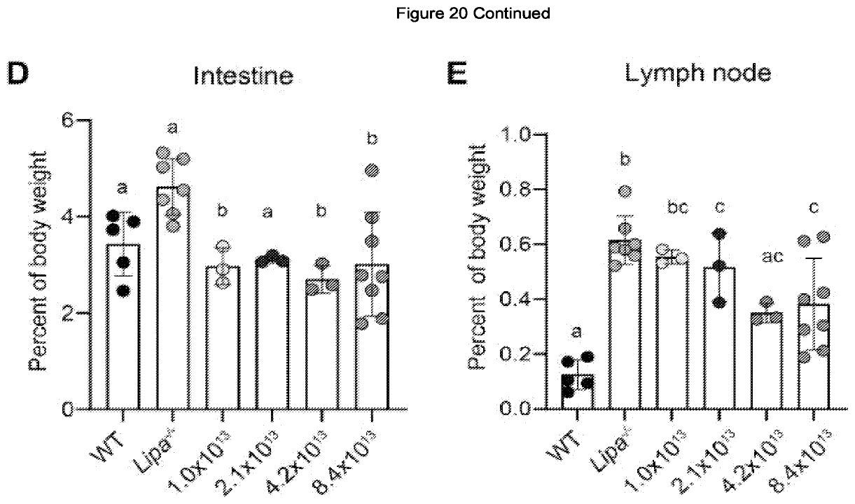

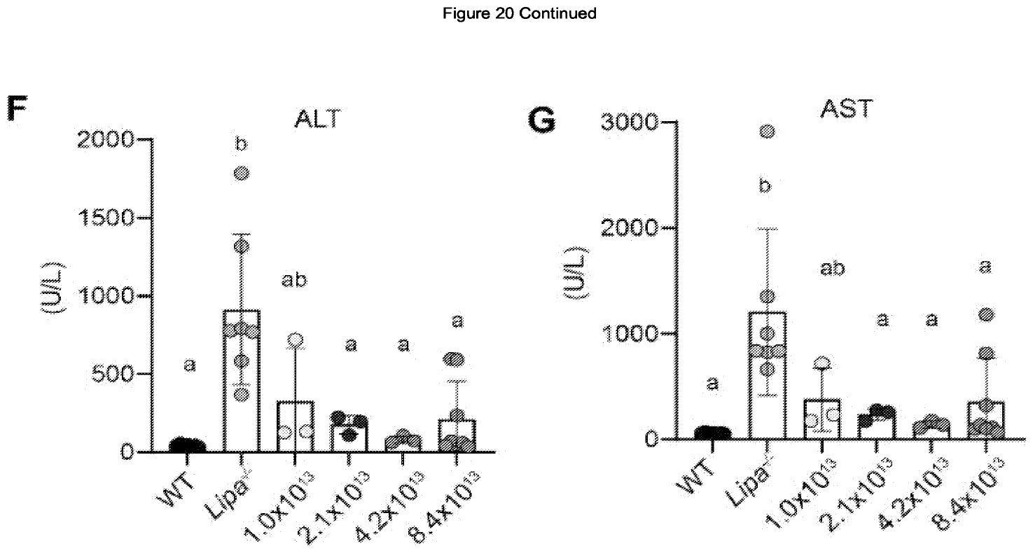

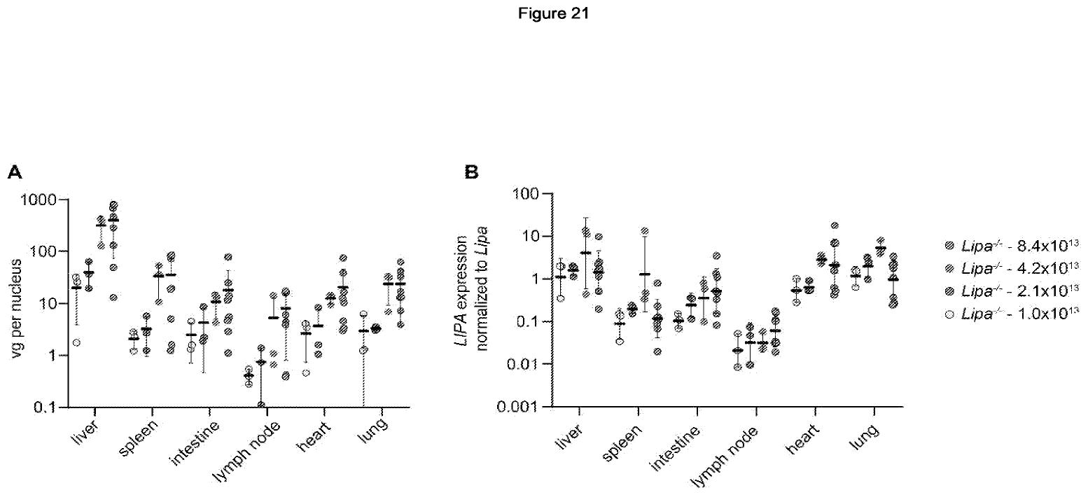

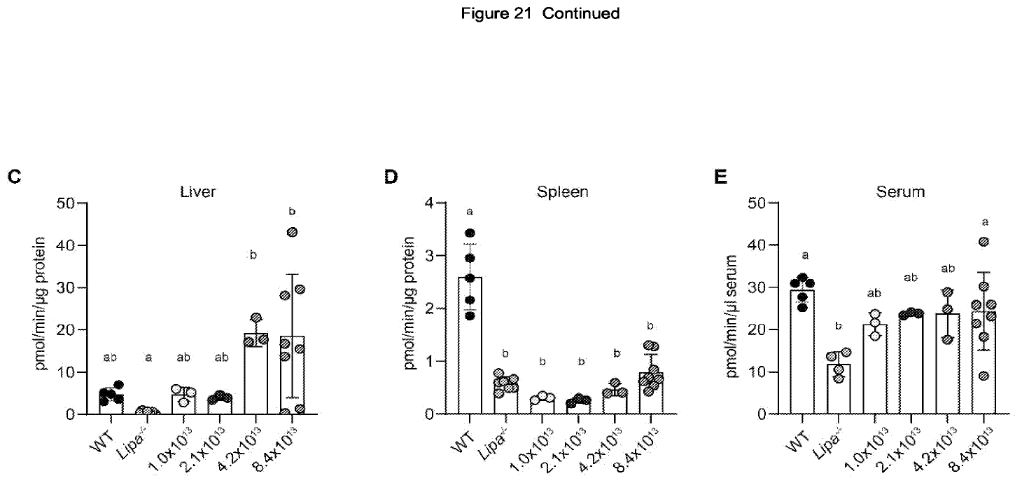

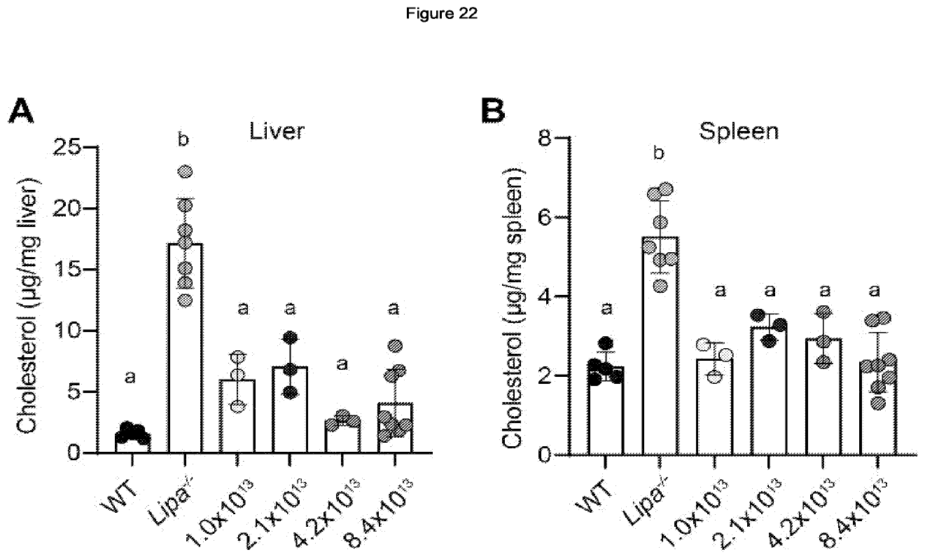

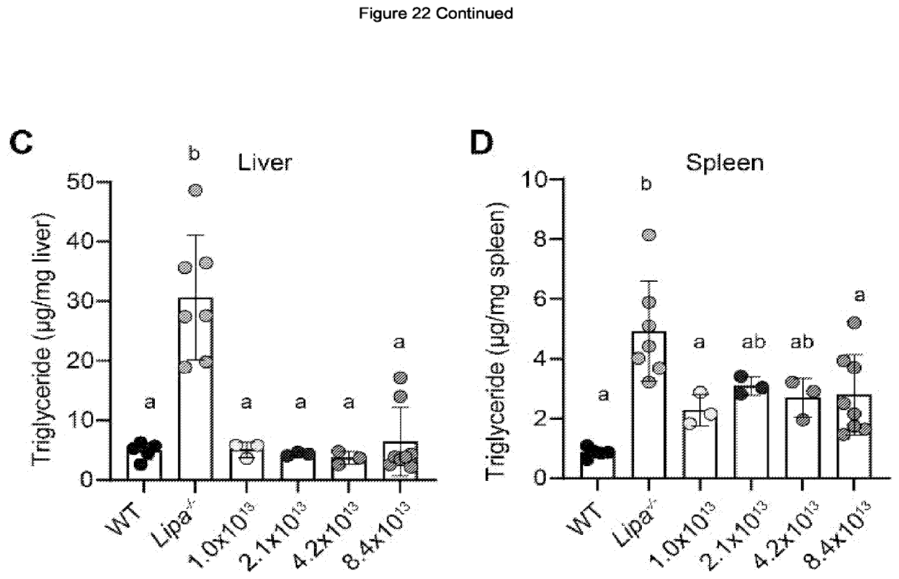

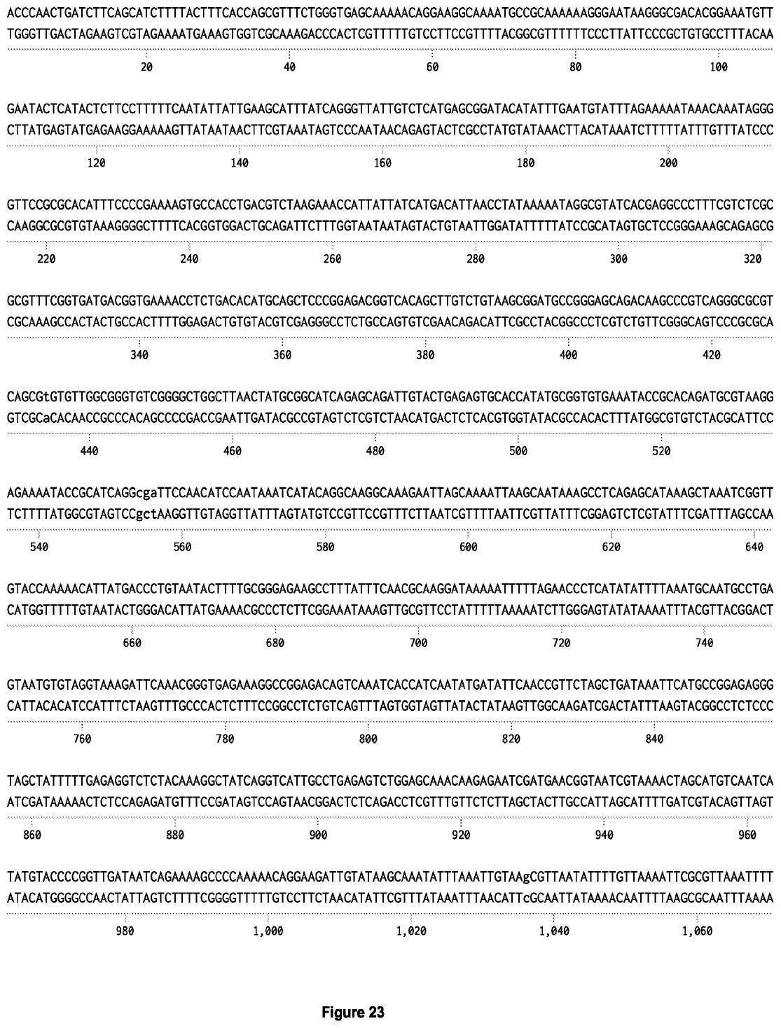

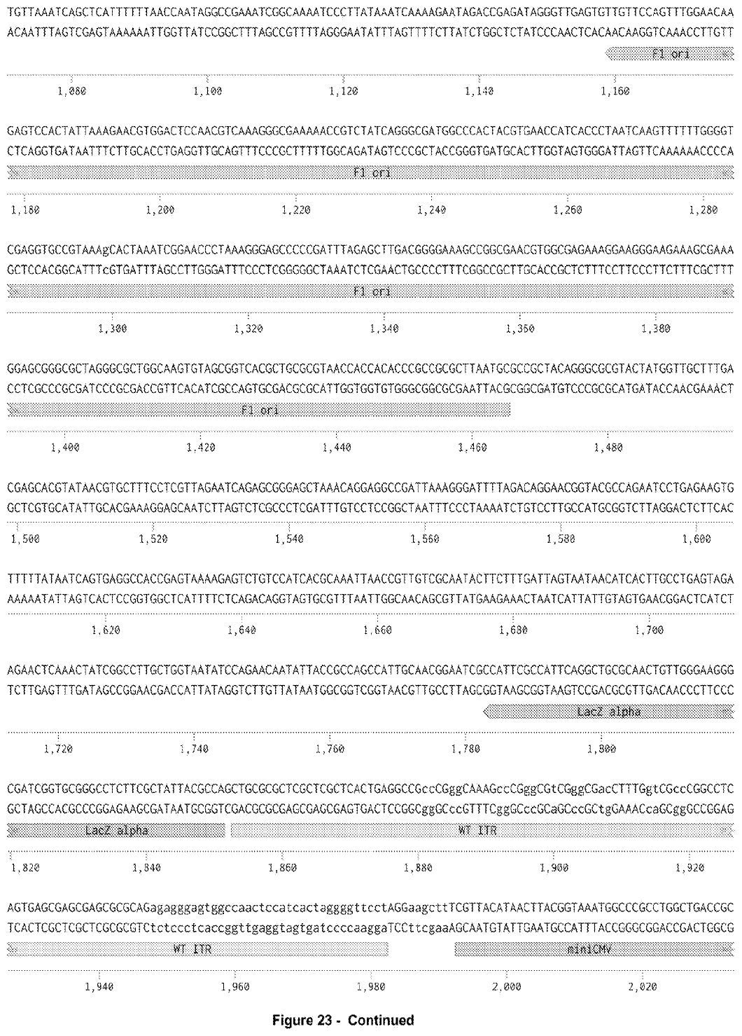

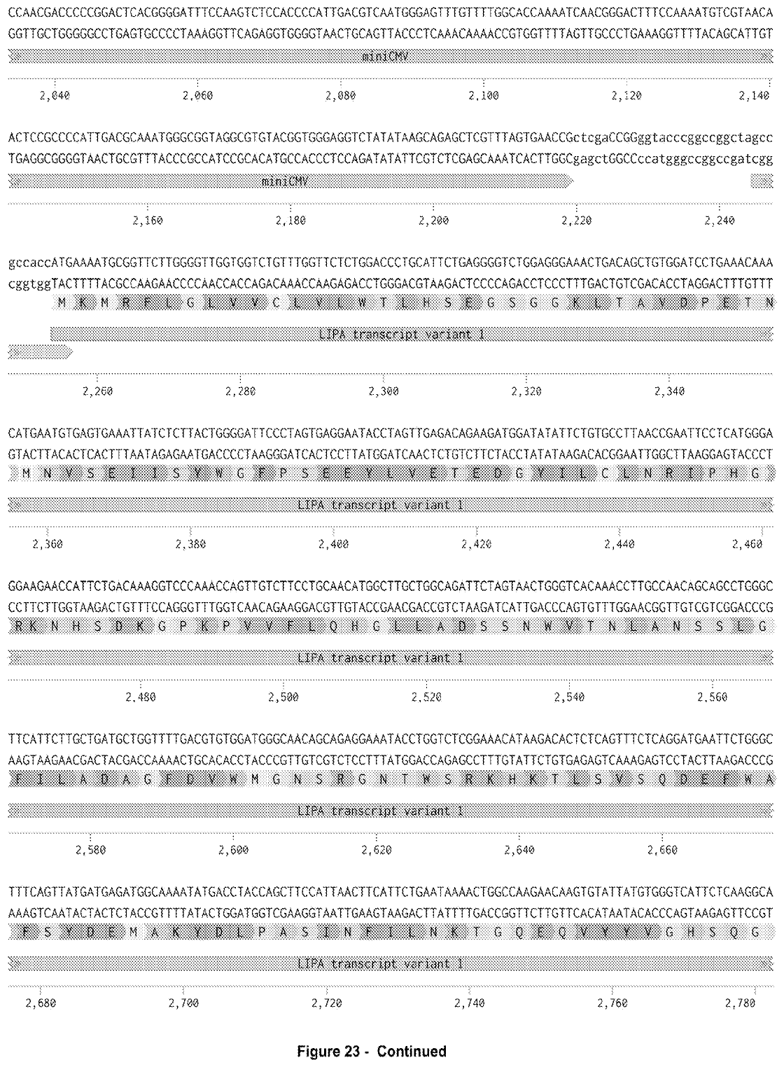

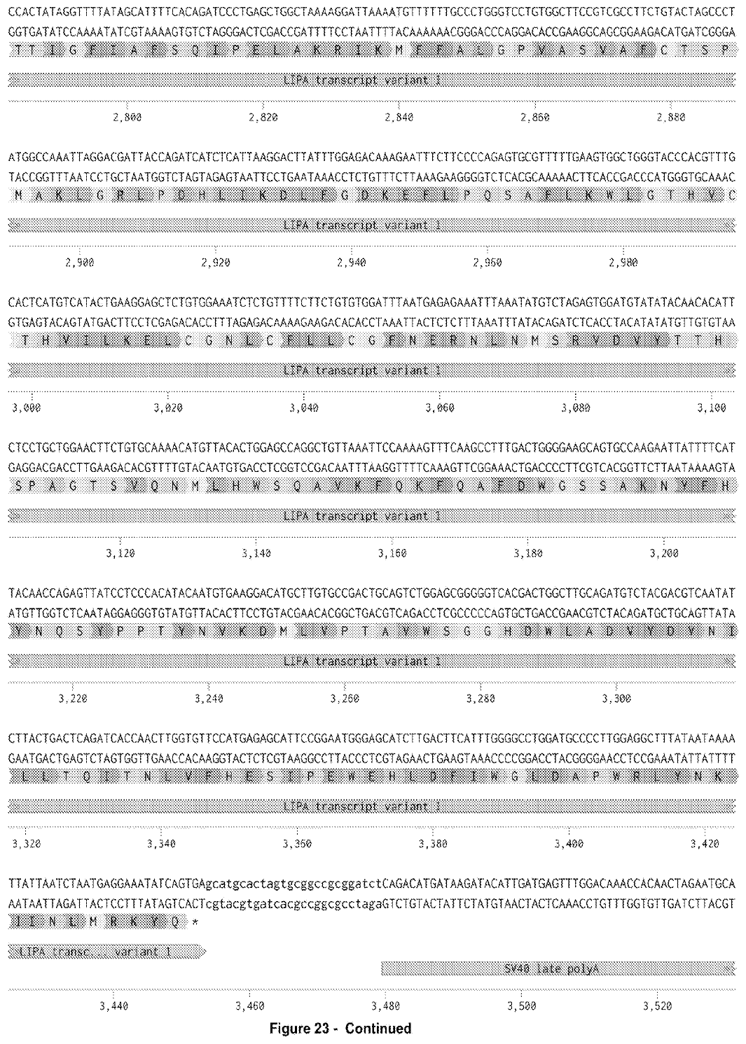

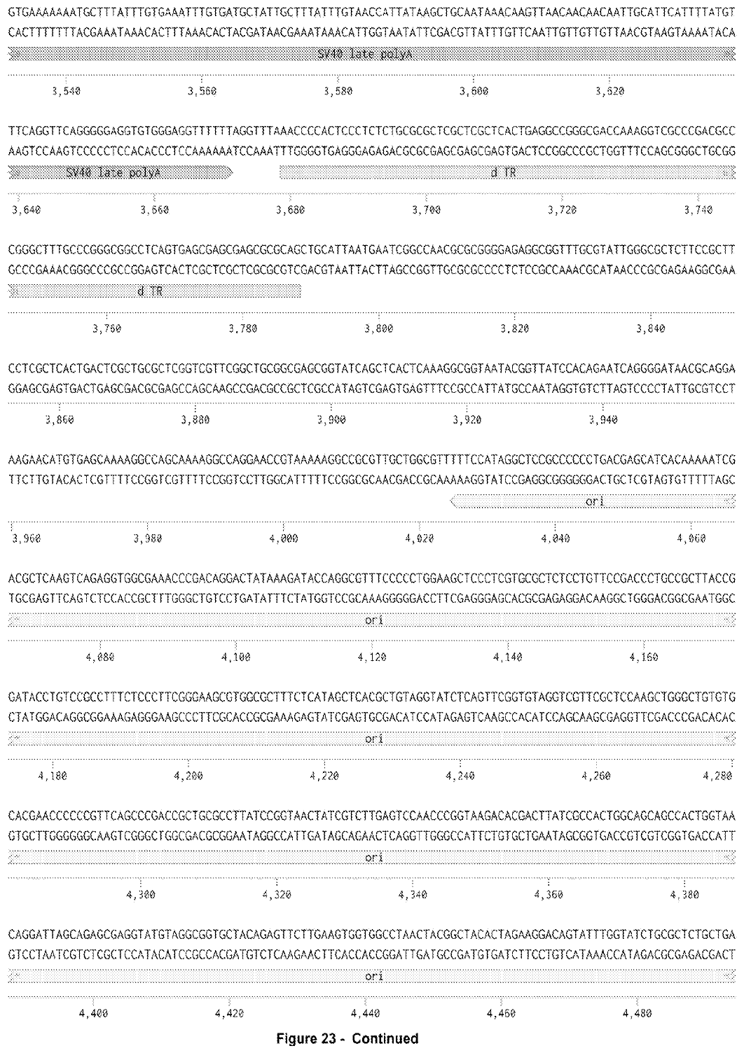

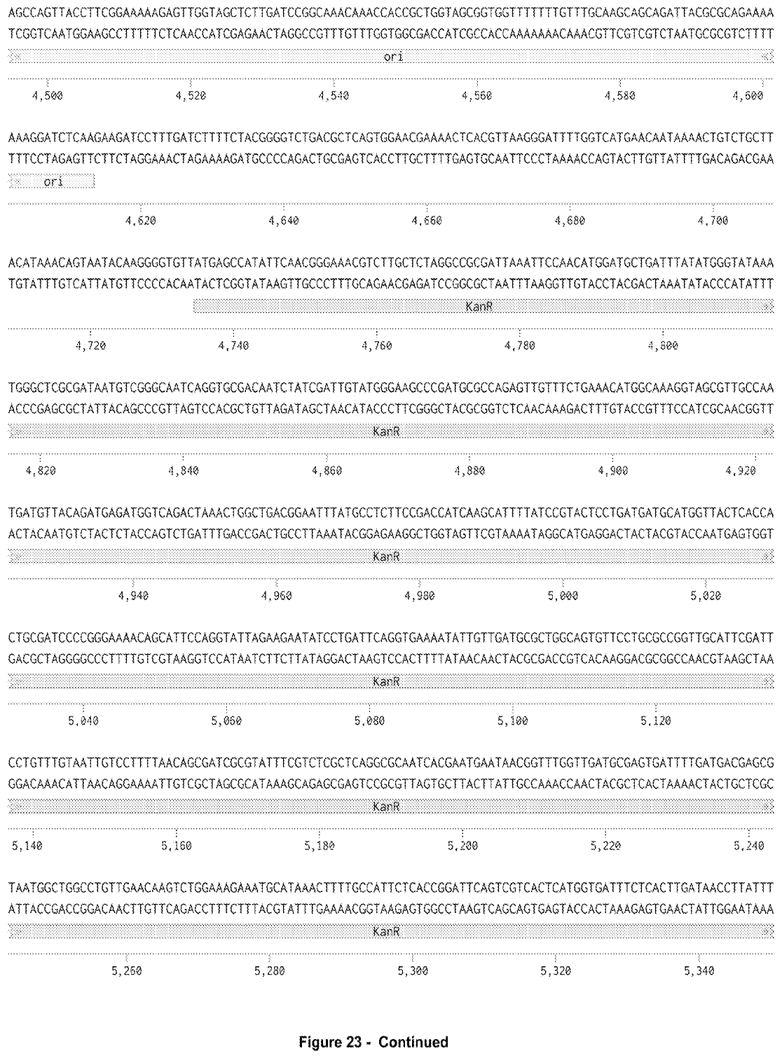

When assayed at the 2-, 4-, and 6-month time points, total cholesterol in the liver of untreated Lipa^{-/-}mice was constantly elevated, on average 19.96±3.97 μg/mg liver, a value approximately 12-fold higher than the WT (FIG. 18). Cholesterol content in the spleen increased more slowly, from 2.65±0.66 μg/mg at 2 months, up to 5.51±0.91 μg/mg at 6 months. Cholesterol content also increased with age in WT spleen (from 1.44±0.70 μg/mg at 2 months to 2.23±0.36 μg/mg at 6 months). Treatment at P2 significantly decreased cholesterol levels in the liver and spleen at 2 months and 4 months post-injection. At 6 months, total cholesterol levels for P2 treatment reverted to untreated Lipa^{-/-}levels in both the liver and the spleen (FIG. 18B), while cholesterol content remained reduced to near WT levels for P60 and P120 injection.

Triglyceride levels in the liver of untreated Lipa^{-/-}mice doubled between 2 months and 4 months of age (14.04±4.15 μg/mg to 29.32±3.70 μg/mg) and then remained constant at 6 months (30.66±10.45 μg/mg) (FIG. 18C). Thus, at 6 months, these values, on average, were elevated 6-fold compared to WT (average 4.70±0.34 μg/mg). At all time points, treatment reduced liver triglycerides significantly, approaching or reaching WT-levels (FIG. 18C). In the spleen, triglyceride content in Lipa^{-/-}mice increased more gradually between 2 months and 6 months of age (2.27±1.32 μg/mg to 4.31±2.82 μg/mg), and it was not until 6 months of age that Lipa^{-/-}spleen triglyceride content was significantly greater than WT. Unlike liver, treatment at P2 and P60 did not significantly alter triglyceride content in the spleen at the 2 month, 4 month, or 6 month endpoint (FIG. 18D). Instead, only treatment at P120 showed a significant decrease.

Serum changes in Lipa^{-/-}mice included reduced total cholesterol, triglycerides, HDL cholesterol, and free fatty acids, along with elevated LDL cholesterol (FIG. 18E-I). Much as seen with lipid levels in the liver and spleen, P2 treatment did not significantly ameliorate any of these changed serum lipid levels at the 2-, 4- or 6-month endpoint, but there were often trends toward improvement. By contrast, treatment at P60 and P120 offset reductions in total cholesterol, HDL cholesterol and free fatty acids in Lipa^{-/-}mice, generating levels near those found in WT mice at the 6-month endpoint. Similarly, treatment partially corrected triglycerides and LDL cholesterol.

Oil Red O (ORO) staining of tissue sections was used to visualize neutral lipids in the liver, spleen, and intestine (FIG. 18J). Compared to the WT, there were large accumulations of lipids stained with ORO in untreated Lipa^{-/-}liver, spleen, and intestine, with almost ubiquitous strong staining being present in the liver. With treatment at all time points, there was a decrease in overall ORO staining, but this was most pronounced for treatment at P60 and P120. After treatment, ORO staining appeared primarily as lipid islands within the tissue, with the more diffuse staining seen in untreated Lipa^{-/-}mice being absent from the remainder of the section.

### Example 6

### LIPA Expression Reduces Macrophages in the Liver

To determine the extent of liver hepatocyte expression in treated livers and the extent of macrophage occupancy by performing LIPA and CD68 immunostaining. 10 μm frozen tissue sections were prepared from liver samples. Slides were fixed in acetone for 10 min at −20° C., allowed to air dry to evaporate excess acetone and washed 2× in PBS. Slides were incubated in BLOXALL Endogenous Blocking Solution (Vector Laboratories) for 10 minutes, washed 2× in PBS, then blocked in 2.5% normal serum for 30 minutes. Slides were incubated overnight at 4° C. with antibodies to LIPA (1:1000; HPA057052, Sigma Aldrich), or CD68 (1:500; MCA1957GA, Bio-Rad). Following incubation, slides were washed 2× in PBS and incubated with ImmPRESS HRP Reagent (Vector Laboratories) for 30 minutes. Slides were then washed 2× in PBS and stained with ImmPACT DAB staining solution (Vector Laboratories) for 5 minutes. They were then counterstained with Hematoxylin QS (Vector Laboratories), dehydrated, differentiated, and mounted in Cytoseal XYL (Thermo Fisher Scientific) with a glass cover slip.

As shown in FIG. 19, at the 6-month endpoint, P2-treated Lipa^{-/-}mice showed only very diffuse and weak staining in liver cells, with little to no punctate staining that would be expected for localization of protein to lysosomes. By contrast, mice injected at P60 and P120 showed increased punctate immunostaining that was evident throughout liver sections. Anti- CD68 staining, a marker used to detect macrophages and Kupffer cells, showed highly elevated staining in untreated Lipa^{-/-}mice. With treatment at all time points, there was a decrease in CD68-positive macrophages, though staining remained within lipid islands present in the liver. Thus, LIPA and CD68 immunostaining supported the notion that P60 and P120 treatment led to high levels of LIPA expression in the liver and to a lowered macrophage occupancy.

### Example 7

### Lower Doses of rscAAVrh74.miniCMV.LIPA Still Show Therapeutic Benefits

A dose of 8.4×10¹³ vg/kg was administered to ensure saturation to determine if gene therapy was feasible for LAL-D. Given the promising data from this dosage, an experiment was designed to determine if lower doses of the gene therapy vector would still prove efficacious. Since injection at P60 (mid-stage) disease provided the most positive results with the high dose, this injection protocol was repeated with 4.2×10¹³, 2.1×10¹³, and 1.05×10¹³ vg/kg of rscAAVrh74.miniCMV.LIPA, ultimately lowering dose 8-fold relative to our starting dose. These mice were followed until 6 months of age (4 months post-injection).

The gross pathology of the liver and spleen was first examined. With decreasing dose, the liver progressively appeared larger and more yellowed, indicating increased fat deposition (FIG. 20A). Nevertheless, treatment at all four doses showed more normal liver appearance when compared to untreated Lipa^{-/-}mice (FIG. 20A), with the two higher doses (8.4×10¹³ and 4.2×10¹³ vg/kg) having slightly more effect than the two lower doses. The spleen also appeared longer and thicker as dose decreased, but again it remained far smaller and less fattened than would occur without treatment (FIG. 20A). Hepatosplenomegaly was reduced at all four doses; the percent of body weight for the liver and the spleen with all 4 doses was significantly reduced relative to untreated Lipa^{-/-}mice (FIG. 20B, C). There was, however, an apparent increase in relative weight as at the two lowest dosages used (1.05×10¹³ vg/kg and 2.1×10¹³ vg/kg) relative to 4.2×10¹³ vg/kg. Likewise, all four doses showed a reduction in the weight of the intestine (FIG. 20D), while only the two highest doses showed a reduction in the weight of the mesenteric lymph node (FIG. 20E). Serum ALT and AST values were significantly decreased at all four doses, again with a slight trend toward higher levels at the lower doses (FIG. 20F and G).

Biodistribution of AAV was examined in all organs and tissues for the dosing experiment (FIG. 21A, Table 5). There was a 2-fold decrease in AAV between doses 8.4×10¹³ vg/kg vs 4.2×10¹³ vg/kg, and 2.1×10¹³ vg/kg vs 1.05×10¹³ vg/kg, while there was a steeper than 2-fold decrease in AAV vgs/nucleus between doses of 4.2×10¹³ vg/kg and 2.4×10¹³ vg/kg in all organs. Most importantly, at the lowest dose of 1.05×10¹³ vg/kg, there was still approximately 20 vg/nucleus in the liver and 2 vg/nucleus in spleen. Interestingly, this translated to less expected human LIPA transcript expression (FIG. 21B); in the liver, at the highest dose (8.4×10¹³ vg/kg), there was only about a 2-fold increase in LIPA expression, normalized to endogenous mouse wild type Lipa expression, despite there being more than 200 vg/nucleus. Using the lowest dose (1.05×10¹³ vg/kg), LIPA transcript levels were still restored to WT levels (FIG. 21B), resulting in WI-levels of enzyme activity in the liver (FIG. 21C). Low expression of AAV and LIPA transcript at all doses in the spleen did not lead to significantly increased enzyme activity relative to untreated Lipa^{-/-}mice (FIG. 21D). LAL enzyme activity in serum was elevated relative to untreated Lipa^{-/-}at all four doses used (FIG. 21E).

TABLE 5
Biodistribution of AAV at the various doses, treated
at P60. Values represent mean vg/nucleus ± SD.

| | 1.05 × 10¹³ | 2.1 × 10¹³ | 4.2 × 10¹³ | 8.4 × 10¹³ |
|---|---|---|---|---|
| | vg/kg | vg/kg | vg/kg | vg/kg |
| | (n = 3) | (n = 3) | (n = 3) | (n = 8) |
| Brain | 0.23 ± 0.11 | 0.77 ± 0.17 | 1.46 ± 0.62 | 2.55 ± 0.81 |
| Heart | 2.69 ± 1.13 | 3.70 ± 2.38 | 12.51 ± 1.55 | 20.52 ± 8.79 |
| Intestines | 2.50 ± 1.03 | 4.29 ± 2.21 | 10.72 ± 3.26 | 17.93 ± 9.08 |
| Liver | 20.05 ± 9.32 | 39.99 ± 12.80 | 316.38 ± 95.55 | 406.13 ± 117.59 |
| Lung | 2.95 ± 1.68 | 3.30 ± 0.15 | 23.89 ± 8.48 | 23.91 ± 7.15 |
| Lymph node | 0.40 ± 0.08 | 0.75 ± 0.37 | 5.32 ± 4.45 | 8.14 ± 2.59 |
| L kidney | 5.91 ± 3.28 | 10.94 ± 4.56 | 24.38 ± 3.99 | 34.95 ± 11.97 |
| R kidney | 5.99 ± 3.81 | 9.23 ± 2.65 | 44.94 ± 10.31 | 30.66 ± 10.44 |
| Spleen | 2.10 ± 0.47 | 3.24 ± 1.32 | 33.41 ± 12.28 | 35.66 ± 13.19 |
| Thymus | 2.41 ± 1.53 | 6.73 ± 1.84 | 74.99 ± 2.58 | 49.44 ± 14.81 |
| L gastrocnemius | 1.11 ± 0.37 | 2.66 ± 1.21 | 6.97 ± 2.76 | 10.75 ± 2.66 |
| R gastrocnemius | 1.89 ± 0.53 | 2.54 ± 0.73 | 12.25 ± 4.62 | 10.79 ± 4.61 |
| L quadricep | 0.70 ± 0.25 | 1.54 ± 0.61 | 9.26 ± 3.41 | 7.11 ± 1.48 |
| R quadricep | 0.48 ± 0.10 | 1.37 ± 0.67 | 6.18 ± 4.14 | 7.50 ± 3.29 |

All four doses also significantly reduced cholesterol (FIG. 22A, B) and triglyceride (FIG. 22C, D) content in liver relative to untreated Lipa^{-/-}mice. Both liver and spleen cholesterol and triglyceride content were reduced to be equivalent to or nearly equivalent to WT levels at all four doses used. Spleen triglyceride levels were reduced less than triglyceride levels in the liver, though all measures were significantly different. Thus, reductions in liver and spleen lipid content can be achieved in Lipa^{-/-}mice with a dose of rscAAVrh74.miniCMV.LIPA as low as 1.05×10¹³ vg/kg.

### REFERENCES

1. Pastores, GM, and Hughes, DA (2020). Lysosomal Acid Lipase Deficiency: Therapeutic Options. Drug Des Devel Ther 14: 591-601.
2. Gomaraschi, M, Bonacina, F, and Norata, GD (2019). Lysosomal Acid Lipase: From Cellular Lipid Handler to Immunometabolic Target. Trends Pharmacol Sci 40: 104-115.
3. Li, F, and Zhang, H (2019). Lysosomal Acid Lipase in Lipid Metabolism and Beyond. Arterioscler Thromb Vasc Biol 39: 850-856.
4. Aguisanda, F, Thorne, N, and Zheng, W (2017). Targeting Wolman Disease and Cholesteryl Ester Storage Disease: Disease Pathogenesis and Therapeutic Development. Curr Chem Genom Transl Med 11: 1-18.
5. Abramov, A, Schorr, S, and Wolman, M (1956). Generalized xanthomatosis with calcified adrenals. AMA J Dis Child 91: 282-286.
6. Burton, BK, Deegan, PB, Enns, GM, Guardamagna, O, Horslen, S, Hovingh, GK, et al. (2015). Clinical Features of Lysosomal Acid Lipase Deficiency. J Pediatr Gastroenterol Nutr 61: 619-625.
7. Pericleous, M, Kelly, C, Wang, T, Livingstone, C, and Ala, A (2017). Wolman's disease and cholesteryl ester storage disorder: the phenotypic spectrum of lysosomal acid lipase deficiency. Lancet Gastroenterol Hepatol 2: 670-679.
8. Shan, Z, and Ju, C (2020). Hepatic Macrophages in Liver Injury. Front Immunol 11: 322.
9. Baratta, F, Pastori, D, Ferro, D, Carluccio, G, Tozzi, G, Angelico, F, et al. (2019). Reduced lysosomal acid lipase activity: A new marker of liver disease severity across the clinical continuum of non-alcoholic fatty liver disease? World J Gastroenterol 25: 4172-4180.
10. Du, H, and Grabowski, GA (2004). Lysosomal acid lipase and atherosclerosis. Curr Opin Lipidol 15: 539-544.
11. Wild, PS, Zeller, T, Schillert, A, Szymczak, S, Sinning, CR, Deiseroth, A, et al. (2011). A genome-wide association study identifies LIPA as a susceptibility gene for coronary artery disease. Circ Cardiovasc Genet 4: 403-412.
12. Burton, BK, Balwani, M, Feillet, F, Baric, I, Burrow, TA, Camarena Grande, C, et al. (2015). A Phase 3 Trial of Sebelipase Alfa in Lysosomal Acid Lipase Deficiency. N Engl J Med 373: 1010-1020.
13. Spencer, HT, Riley, BE, and Doering, CB (2016). State of the art: gene therapy of haemophilia. Haemophilia 22 Suppl 5: 66-71.
14. High, KA (2012). The gene therapy journey for hemophilia: are we there yet? Hematology Am Soc Hematol Educ Program 2012: 375-381.
15. Rodino-Klapac, LR, Chicoine, LG, Kaspar, BK, and Mendell, JR (2007). Gene therapy for duchenne muscular dystrophy: expectations and challenges. Arch Neurol 64: 1236-1241.
16. Mendell, JR, Al-Zaidy, S, Shell, R, Arnold, WD, Rodino-Klapac, LR, Prior, TW, et al. (2017). Single-Dose Gene-Replacement Therapy for Spinal Muscular Atrophy. N Engl J Med 377: 1713-1722.
17. Testa, F, Maguire, AM, Rossi, S, Pierce, EA, Melillo, P, Marshall, K, et al. (2013). Three-year follow-up after unilateral subretinal delivery of adeno-associated virus in patients with Leber congenital Amaurosis type 2. Ophthalmology 120: 1283-1291.
18. Nagree, MS, Scalia, S, McKillop, WM, and Medin, JA (2019). An update on gene therapy for lysosomal storage disorders. Expert Opin Biol Ther 19: 655-670.
19. Penati, R, Fumagalli, F, Calbi, V, Bernardo, ME, and Aiuti, A (2017). Gene therapy for lysosomal storage disorders: recent advances for metachromatic leukodystrophy and mucopolysaccaridosis I. J Inherit Metab Dis 40: 543-554.
20. Biffi, A (2016). Gene therapy for lysosomal storage disorders: a good start. Hum Mol Genet 25: R65-75.
21. Rastall, DP, and Amalfitano, A (2015). Recent advances in gene therapy for lysosomal storage disorders. Appl Clin Genet 8: 157-169.
22. Zygmunt, DA, Xu, R, Jia, Y, Ashbrook, A, Menke, C, Shao, G, et al. (2019). rAAVrh74.MCK.GALGT2 Demonstrates Safety and Widespread Muscle Glycosylation after Intravenous Delivery in C57BL/6J Mice. Mol Ther Methods Clin Dev 15: 305-319.
23. Chicoine, LG, Rodino-Klapac, LR, Shao, G, Xu, R, Bremer, WG, Camboni, M, et al. (2014). Vascular delivery of rAAVrh74.MCK.GALGT2 to the gastrocnemius muscle of the rhesus macaque stimulates the expression of dystrophin and laminin alpha2 surrogates. Mol Ther 22: 713-724.
24. Martin, PT, Xu, R, Rodino-Klapac, LR, Oglesbay, E, Camboni, M, Montgomery, CL, et al. (2009). Overexpression of Galgt2 in skeletal muscle prevents injury resulting from eccentric contractions in both mdx and wild-type mice. Am J Physiol Cell Physiol 296: C476-488.
25. Grieger, JC, and Samulski, RJ (2005). Adeno-associated virus as a gene therapy vector: vector development, production and clinical applications. Adv Biochem Eng Biotechnol 99: 119-145.
26. Rosas, LE, Grieves, JL, Zaraspe, K, La Perle, KM, Fu, H, and McCarty, DM (2012). Patterns of scAAV vector insertion associated with oncogenic events in a mouse model for genotoxicity. Mol Ther 20: 2098-2110.
27. Xiao, X, Li, J, and Samulski, RJ (1998). Production of high-titer recombinant adeno-associated virus vectors in the absence of helper adenovirus. J Virol 72: 2224-2232.
28. Bish, LT, Morine, K, Sleeper, MM, Sanmiguel, J, Wu, D, Gao, G, et al. (2008). Adeno-associated virus (AAV) serotype 9 provides global cardiac gene transfer superior to AAV1, AAV6, AAV7, and AAV8 in the mouse and rat. Hum Gene Ther 19: 1359-1368.
29. Cunningham, SC, and Alexander, IE (2019). AAV-Mediated Gene Delivery to the Mouse Liver. Methods Mol Biol 1937: 213-219.
30. Palaschak, B, Herzog, RW, and Markusic, DM (2019). AAV-Mediated Gene Delivery to the Liver: Overview of Current Technologies and Methods. Methods Mol Biol 1950: 333-360.
31. McCarty, DM (2008). Self-complementary AAV vectors; advances and applications. Mol Ther 16: 1648-1656.
32. Fu, H, Zaraspe, K, Murakami, N, Meadows, AS, Pineda, RJ, McCarty, DM, et al. (2018). Targeting Root Cause by Systemic scAAV9-hIDS Gene Delivery: Functional Correction and Reversal of Severe MPS II in Mice. Mol Ther Methods Clin Dev 10: 327-340.
33. Du, H, Duanmu, M, Witte, D, and Grabowski, GA (1998). Targeted disruption of the mouse lysosomal acid lipase gene: long-term survival with massive cholesteryl ester and triglyceride storage. Hum Mol Genet 7: 1347-1354.
34. Du, H, Heur, M, Duanmu, M, Grabowski, GA, Hui, DY, Witte, DP, et al. (2001). Lysosomal acid lipase-deficient mice: depletion of white and brown fat, severe hepatosplenomegaly, and shortened life span. J Lipid Res 42: 489-500.
35. Sun, Y, Xu, YH, Du, H, Quinn, B, Liou, B, Stanton, L, et al. (2014). Reversal of advanced disease in lysosomal acid lipase deficient mice: a model for lysosomal acid lipase deficiency disease. Mol Genet Metab 112: 229-241.
36. Mendell JR, Al-Zaidy S, Shell R, Arnold WD, Rodino-Klapac LR, Prior TW, et al. Single-Dose Gene-Replacement Therapy for Spinal Muscular Atrophy. New England Journal of Medicine. 2017;377(18):1713-22.
37. Mendell JR, Sahenk Z, Lehman K, Nease C, Lowes LP, Miller NF, et al. Assessment of Systemic Delivery of rAAVrh74.MHCK7.micro-dystrophin in Children With Duchenne Muscular Dystrophy: A Nonrandomized Controlled Trial. JAMA Neurology.

LIPA gene variant 1
SEQ ID NO: 1
ATGAAAATGCGGTTCTTGGGGTTGGTGGTCTGTTTGGTTCTCTGGACCCTGCATTCTGA
GGGGTCTGGAGGGAAACTGACAGCTGTGGATCCTGAAACAAACATGAATGTGAGTGAA
ATTATCTCTTACTGGGGATTCCCTAGTGAGGAATACCTAGTTGAGACAGAAGATGGATA
TATTCTGTGCCTTAACCGAATTCCTCATGGGAGGAAGAACCATTCTGACAAAGGTCCCA
AACCAGTTGTCTTCCTGCAACATGGCTTGCTGGCAGATTCTAGTAACTGGGTCACAAAC
CTTGCCAACAGCAGCCTGGGCTTCATTCTTGCTGATGCTGGTTTTGACGTGTGGATGG
GCAACAGCAGAGGAAATACCTGGTCTCGGAAACATAAGACACTCTCAGTTTCTCAGGAT
GAATTCTGGGCTTTCAGTTATGATGAGATGGCAAAATATGACCTACCAGCTTCCATTAAC
TTCATTCTGAATAAAACTGGCCAAGAACAAGTGTATTATGTGGGTCATTCTCAAGGCAC
CACTATAGGTTTTATAGCATTTTCACAGATCCCTGAGCTGGCTAAAAGGATTAAAATGTT
TTTTGCCCTGGGTCCTGTGGCTTCCGTCGCCTTCTGTACTAGCCCTATGGCCAAATTAG
GACGATTACCAGATCATCTCATTAAGGACTTATTTGGAGACAAAGAATTTCTTCCCCAGA
GTGCGTTTTTGAAGTGGCTGGGTACCCACGTTTGCACTCATGTCATACTGAAGGAGCTC
TGTGGAAATCTCTGTTTTCTTCTGTGTGGATTTAATGAGAGAAATTTAAATATGTCTAGA
GTGGATGTATATACAACACATTCTCCTGCTGGAACTTCTGTGCAAAACATGTTACACTG
GAGCCAGGCTGTTAAATTCCAAAAGTTTCAAGCCTTTGACTGGGGAAGCAGTGCCAAG
AATTATTTTCATTACAACCAGAGTTATCCTCCCACATACAATGTGAAGGACATGCTTGTG
CCGACTGCAGTCTGGAGCGGGGGTCACGACTGGCTTGCAGATGTCTACGACGTCAATA
TCTTACTGACTCAGATCACCAACTTGGTGTTCCATGAGAGCATTCCGGAATGGGAGCAT
CTTGACTTCATTTGGGGCCTGGATGCCCCTTGGAGGCTTTATAATAAAATTATTAATCTA
ATGAGGAAATATCAGTGA
SEQ ID NO: 3
TCGTTACATAACTTACGGTAAATGGCCCGCCTGGCTGACCGCCCAACGACCCCCGGAC
TCACGGGGATTTCCAAGTCTCCACCCCATTGACGTCAATGGGAGTTTGTTTTGGCACCA
AAATCAACGGGACTTTCCAAAATGTCGTAACAACTCCGCCCCATTGACGCAAATGGGCG
GTAGGCGTGTACGGTGGGAGGTCTATATAAGCAGAGCTCGTTTAGTGAACCG
ptrs.miniCMV.LIPA AAV vector plasmid sequence
(SEQ ID NO: 4)
ACCCAACTGATCTTCAGCATCTTTTACTTTCACCAGCGTTTCTGGGTGAGCAAAAACAG
GAAGGCAAAATGCCGCAAAAAAGGGAATAAGGGCGACACGGAAATGTTGAATACTCAT
ACTCTTCCTTTTTCAATATTATTGAAGCATTTATCAGGGTTATTGTCTCATGAGCGGATAC
ATATTTGAATGTATTTAGAAAAATAAACAAATAGGGGTTCCGCGCACATTTCCCCGAAAA
GTGCCACCTGACGTCTAAGAAACCATTATTATCATGACATTAACCTATAAAAATAGGCGT
ATCACGAGGCCCTTTCGTCTCGCGCGTTTCGGTGATGACGGTGAAAACCTCTGACACA
TGCAGCTCCCGGAGACGGTCACAGCTTGTCTGTAAGCGGATGCCGGGAGCAGACAAG
CCCGTCAGGGCGCGTCAGCGtGTGTTGGCGGGTGTCGGGGCTGGCTTAACTATGCGG
CATCAGAGCAGATTGTACTGAGAGTGCACCATATGCGGTGTGAAATACCGCACAGATG
CGTAAGGAGAAAATACCGCATCAGGcgaTTCCAACATCCAATAAATCATACAGGCAAGG
CAAAGAATTAGCAAAATTAAGCAATAAAGCCTCAGAGCATAAAGCTAAATCGGTTGTAC
CAAAAACATTATGACCCTGTAATACTTTTGCGGGAGAAGCCTTTATTTCAACGCAAGGAT
AAAAATTTTTAGAACCCTCATATATTTTAAATGCAATGCCTGAGTAATGTGTAGGTAAAG
ATTCAAACGGGTGAGAAAGGCCGGAGACAGTCAAATCACCATCAATATGATATTCAACC
GTTCTAGCTGATAAATTCATGCCGGAGAGGGTAGCTATTTTTGAGAGGTCTCTACAAAG
GCTATCAGGTCATTGCCTGAGAGTCTGGAGCAAACAAGAGAATCGATGAACGGTAATC
GTAAAACTAGCATGTCAATCATATGTACCCCGGTTGATAATCAGAAAAGCCCCAAAAAC
AGGAAGATTGTATAAGCAAATATTTAAATTGTAAgCGTTAATATTTTGTTAAAATTCGCGT
TAAATTTTTGTTAAATCAGCTCATTTTTTAACCAATAGGCCGAAATCGGCAAAATCCCTTA
TAAATCAAAAGAATAGACCGAGATAGGGTTGAGTGTTGTTCCAGTTTGGAACAAGAGTC
CACTATTAAAGAACGTGGACTCCAACGTCAAAGGGCGAAAAACCGTCTATCAGGGCGA
TGGCCCACTACGTGAACCATCACCCTAATCAAGTTTTTTGGGGTCGAGGTGCCGTAAAg
CACTAAATCGGAACCCTAAAGGGAGCCCCCGATTTAGAGCTTGACGGGGAAAGCCGGC
GAACGTGGCGAGAAAGGAAGGGAAGAAAGCGAAAGGAGCGGGCGCTAGGGCGCTGG
CAAGTGTAGCGGTCACGCTGCGCGTAACCACCACACCCGCCGCGCTTAATGCGCCGC
TACAGGGCGCGTACTATGGTTGCTTTGACGAGCACGTATAACGTGCTTTCCTCGTTAGA
ATCAGAGCGGGAGCTAAACAGGAGGCCGATTAAAGGGATTTTAGACAGGAACGGTACG
CCAGAATCCTGAGAAGTGTTTTTATAATCAGTGAGGCCACCGAGTAAAAGAGTCTGTCC
ATCACGCAAATTAACCGTTGTCGCAATACTTCTTTGATTAGTAATAACATCACTTGCCTG
AGTAGAAGAACTCAAACTATCGGCCTTGCTGGTAATATCCAGAACAATATTACCGCCAG
CCATTGCAACGGAATCGCCATTCGCCATTCAGGCTGCGCAACTGTTGGGAAGGGCGAT
CGGTGCGGGCCTCTTCGCTATTACGCCAGCTGCGCGCTCGCTCGCTCACTGAGGCCG
ccCGggCAAAGccCGggCGtCGggCGacCTTTGgtCGccCGGCCTCAGTGAGCGAGCGAGC
GCGCAGagagggagtggccaactccatcactaggggttcctAGGaagcttTCGTTACATAACTTACGGTAA
ATGGCCCGCCTGGCTGACCGCCCAACGACCCCCGGACTCACGGGGATTTCCAAGTCT
CCACCCCATTGACGTCAATGGGAGTTTGTTTTGGCACCAAAATCAACGGGACTTTCCAA
AATGTCGTAACAACTCCGCCCCATTGACGCAAATGGGGGGTAGGCGTGTACGGTGGGA
GGTCTATATAAGCAGAGCTCGTTTAGTGAACCGctcgaCCGGggtacccggccggctagccgccacc
ATGAAAATGCGGTTCTTGGGGTTGGTGGTCTGTTTGGTTCTCTGGACCCTGCATTCTGA
GGGGTCTGGAGGGAAACTGACAGCTGTGGATCCTGAAACAAACATGAATGTGAGTGAA
ATTATCTCTTACTGGGGATTCCCTAGTGAGGAATACCTAGTTGAGACAGAAGATGGATA
TATTCTGTGCCTTAACCGAATTCCTCATGGGAGGAAGAACCATTCTGACAAAGGTCCCA
AACCAGTTGTCTTCCTGCAACATGGCTTGCTGGCAGATTCTAGTAACTGGGTCACAAAC
CTTGCCAACAGCAGCCTGGGCTTCATTCTTGCTGATGCTGGTTTTGACGTGTGGATGG
GCAACAGCAGAGGAAATACCTGGTCTCGGAAACATAAGACACTCTCAGTTTCTCAGGAT
GAATTCTGGGCTTTCAGTTATGATGAGATGGCAAAATATGACCTACCAGCTTCCATTAAC
TTCATTCTGAATAAAACTGGCCAAGAACAAGTGTATTATGTGGGTCATTCTCAAGGCAC
CACTATAGGTTTTATAGCATTTTCACAGATCCCTGAGCTGGCTAAAAGGATTAAAATGTT
TTTTGCCCTGGGTCCTGTGGCTTCCGTCGCCTTCTGTACTAGCCCTATGGCCAAATTAG
GACGATTACCAGATCATCTCATTAAGGACTTATTTGGAGACAAAGAATTTCTTCCCCAGA
GTGCGTTTTTGAAGTGGCTGGGTACCCACGTTTGCACTCATGTCATACTGAAGGAGCTC
TGTGGAAATCTCTGTTTTCTTCTGTGTGGATTTAATGAGAGAAATTTAAATATGTCTAGA
GTGGATGTATATACAACACATTCTCCTGCTGGAACTTCTGTGCAAAACATGTTACACTG
GAGCCAGGCTGTTAAATTCCAAAAGTTTCAAGCCTTTGACTGGGGAAGCAGTGCCAAG
AATTATTTTCATTACAACCAGAGTTATCCTCCCACATACAATGTGAAGGACATGCTTGTG
CCGACTGCAGTCTGGAGCGGGGGTCACGACTGGCTTGCAGATGTCTACGACGTCAATA
TCTTACTGACTCAGATCACCAACTTGGTGTTCCATGAGAGCATTCCGGAATGGGAGCAT
CTTGACTTCATTTGGGGCCTGGATGCCCCTTGGAGGCTTTATAATAAAATTATTAATCTA
ATGAGGAAATATCAGTGAgcatgcactagtgcggccgcggatctCAGACATGATAAGATACATTGAT
GAGTTTGGACAAACCACAACTAGAATGCAGTGAAAAAAATGCTTTATTTGTGAAATTTGT
GATGCTATTGCTTTATTTGTAACCATTATAAGCTGCAATAAACAAGTTAACAACAACAATT
GCATTCATTTTATGTTTCAGGTTCAGGGGGAGGTGTGGGAGGTTTTTTAGGTTTAAACC
CCACTCCCTCTCTGCGCGCTCGCTCGCTCACTGAGGCCGGGCGACCAAAGGTCGCCC
GACGCCCGGGCTTTGCCCGGGGGCCTCAGTGAGCGAGCGAGCGCGCAGCTGCATT
AATGAATCGGCCAACGCGCGGGGAGAGGCGGTTTGCGTATTGGGCGCTCTTCCGCTT
CCTCGCTCACTGACTCGCTGCGCTCGGTCGTTCGGCTGCGGCGAGCGGTATCAGCTC
ACTCAAAGGCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGAGG
TGGCGAAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCCTTCG
GGAAGCGTGGCGCTTTCTCATAGCTCACGCTGTAGGTATCTCAGTTCGGTGTAGGTCG
TTCGCTCCAAGCTGGGCTGTGTGCACGAACCCCCCGTTCAGCCCGACCGCTGCGCCTT
ATCCGGTAACTATCGTCTTGAGTCCAACCCGGTAAGACACGACTTATCGCCACTGGCA
GCAGCCACTGGTAACAGGATTAGCAGAGCGAGGTATGTAGGCGGTGCTACAGAGTTCT
TGAAGTGGTGGCCTAACTACGGCTACACTAGAAGGACAGTATTTGGTATCTGCGCTCTG
CTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACTCA
CGTTAAGGGATTTTGGTCATGAACAATAAAACTGTCTGCTTACATAAACAGTAATACAAG
GGGTGTTATGAGCCATATTCAACGGGAAACGTCTTGCTCTAGGCCGCGATTAAATTCCA
ACATGGATGCTGATTTATATGGGTATAAATGGGCTCGCGATAATGTCGGGCAATCAGGT
GCGACAATCTATCGATTGTATGGGAAGCCCGATGCGCCAGAGTTGTTTCTGAAACATG
GCAAAGGTAGCGTTGCCAATGATGTTACAGATGAGATGGTCAGACTAAACTGGCTGAC
GGAATTTATGCCTCTTCCGACCATCAAGCATTTTATCCGTACTCCTGATGATGCATGGTT
ACTCACCACTGCGATCCCCGGGAAAACAGCATTCCAGGTATTAGAAGAATATCCTGATT
CAGGTGAAAATATTGTTGATGCGCTGGCAGTGTTCCTGCGCCGGTTGCATTCGATTCCT
GTTTGTAATTGTCCTTTTAACAGCGATCGCGTATTTCGTCTCGCTCAGGCGCAATCACG
AATGAATAACGGTTTGGTTGATGCGAGTGATTTTGATGACGAGCGTAATGGCTGGCCTG
TTGAACAAGTCTGGAAAGAAATGCATAAACTTTTGCCATTCTCACCGGATTCAGTCGTCA
CTCATGGTGATTTCTCACTTGATAACCTTATTTTTGACGAGGGGAAATTAATAGGTTGTA
TTGATGTTGGACGAGTCGGAATCGCAGACCGATACCAGGATCTTGCCATCCTATGGAA
CTGCCTCGGTGAGTTTTCTCCTTCATTACAGAAACGGCTTTTTCAAAAATATGGTATTGA
TAATCCTGATATGAATAAATTGCAGTTTCATTTGATGCTCGATGAGTTTTTCTAAGAATTC
GTTCTTCGGGGCGAAAACTCTCAAGGATCTTACCGCTGTTGAGATCCAGTTCGATGTAA
CCCACTCGTGC

What is claimed is:

1. A polynucleotide comprising
(a) one or more regulatory control elements; and
(b) a lipase A (LIPA) cDNA sequence.

2. The polynucleotide of claim 1, wherein the regulatory control element is a miniCMV promoter or a fragment thereof.

3. The polynucleotide of claim 1 or claim 2, wherein the LIPA cDNA comprises (a) a nucleotide sequence having at least 95% sequence identity to SEQ ID NO: 1 or (b) the nucleotide sequence set forth in SEQ ID NO: 1.

4. The polynucleotide of any one of claims 1-3 further comprising the nucleotide sequence of SEQ ID NO: 3.

5. The polynucleotide of any one of claims 1-3 comprising nucleotides 1853-3906 of SEQ ID NO: 4.

6. The polynucleotide of any of claims 1-5 comprising (a) a nucleotide sequence that is at least 95% identical to SEQ ID NO: 4 or (b) the nucleotide sequence of SEQ ID NO: 4.

7. A recombinant adeno-associated virus (rAAV) having a genome comprising a polynucleotide sequence of any one of claims 1-5.

8. The rAAV of claim 7, wherein the rAAV is of the serotype AAV1, AAV2, AAV3, AAV4, AAV5, AAV6, AAV7, AAV8, AAV9, AAVRH10, AAVrh74, AAV11, AAV12, AAV13 or Anc80, AAV7m8 and their derivatives.

9. An rAAV particle comprising the rAAV of claim 7 or 8.

10. A composition comprising the rAAV of claim 7 or 8 or the viral particle of claim 9.

11. The composition of claim 10, wherein the composition is formulated for intravenous delivery.

12. A method of treating a lysosomal Acid Lipase Deficiency (LAL-D) in a subject in need thereof comprising administering a polynucleotide of any one of claims 1-7, an rAAV of claim 7 or 8, a rAAV particle of claim 9 or a composition of claim 10 or 11.

13. The method of claim 12 wherein the LAL-D is Wolman disease or cholesterol ester storage disease.

14. The method of claim 12 or 13 wherein the subject has a mutation in the LAL-D gene.

15. A method of treating a disorder related to lipid storage or accumulation in a subject in need thereof comprising administering a polynucleotide of any one of claims 1-7, an rAAV of claim 7 or 8, a rAAV particle of claim 9 or a composition of claim 10 or 11.

16. The method of claim 14 wherein the disorder is coronary artery disease, atherosclerosis, type II diabetes, obesity or non-alcoholic fatty liver disease.

17. A method of treating dyslipidemia or hypercholesterolemia in a subject in need thereof comprising administering a polynucleotide of any one of claims 1-7, an rAAV of claim 7 or 8, a rAAV particle of claim 9 or a composition of claim 10 or 11.

18. A method of decreasing triglycerides, cholesterol, and/or fatty acids in a subject in need thereof comprising administering a polynucleotide of any one of claims 1-7, an rAAV of claim 7 or 8, a rAAV particle of claim 9 or a composition of claim 10 or 11.

19. The method of any one of claims 12-18, further comprising a step of administering an immunosuppressing agent.

20. The method of any one of claims 12-19, wherein the polynucleotide, rAAV, rAAV particle or composition are administered by intravenous or intraperitoneal delivery or intraperitoneally.

21. Use of a polynucleotide of any one of claims 1-7, an rAAV of claim 7 or 8, a rAAV particle of claim 9 or a composition of claim 10 or 11 for the preparation of a medicament for the treatment of a lysosomal acid lipase deficiency (LAL-D) in a subject in need thereof.

22. The use of claim 21 wherein the LAL-D is Wolman disease or cholesterol ester storage disease.

23. The use of claim 21 or 22 wherein the subject has a mutation in the LAL-D gene. 24 Use of a polynucleotide of any one of claims 1-7, an rAAV of claim 7 or 8, a rAAV particle of claim 9 or a composition of claim 10 or 11 for the preparation of a medicament for the treatment of a disorder related to lipid storage or accumulation in a subject in need thereof.

25. The use of claim 24 wherein the disorder is coronary artery disease, atherosclerosis, type II diabetes, obesity or non-alcoholic fatty liver disease.

26. Use of a polynucleotide of any one of claims 1-7, an rAAV of claim 7 or 8, a rAAV particle of claim 9 or a composition of claim 10 or 11 for the preparation of a medicament for treating dyslipidemia or hypercholesterolemia in a subject in need thereof.

27. Use of a polynucleotide of any one of claims 1-7, an rAAV of claim 7 or 8, a rAAV particle of claim 9 or a composition of claim 10 or 11 for the preparation of a medicament for decreasing triglycerides, cholesterol, and/or fatty acids in a subject in need thereof.

28. The use of any one of claims 21-27, wherein the medicament is administered with an immunosuppressing agent.

29. The use of any one of claims 21-28, wherein the medicament is formulated for intravenous or intraperitoneal delivery.

30. A composition for treating a lysosomal acid lipase deficiency (LAL-D) in a subject in need thereof comprising, wherein the composition comprises a polynucleotide of any one of claims 1-7, an rAAV of claim 7 or 8, a rAAV particle of claim 9 or a composition of claim 10 or 11.

31. The composition of claim 30 wherein the LAL-D is Wolman disease or cholesterol ester storage disease.

32. The composition of claim 30 or 31 wherein the subject has a mutation in the LAL-D gene.

33. A composition for treating a disorder related to lipid storage or accumulation in a subject in need thereof, wherein the composition comprises a polynucleotide of any one of claims 1-7, an rAAV of claim 7 or 8, a rAAV particle of claim 9 or a composition of claim 10 or 11.

34. The composition of claim 33 wherein the disorder is coronary artery disease, atherosclerosis, type II diabetes, obesity or non-alcoholic fatty liver disease.

35. A composition for treating dyslipidemia or hypercholesterolemia in a subject in need thereof, wherein the composition comprises a polynucleotide of any one of claims 1-7, an rAAV of claim 7 or 8, a rAAV particle of claim 9 or a composition of claim 10 or 11.

36. A composition for decreasing triglycerides, cholesterol, and/or fatty acids in a subject in need thereof, wherein the the composition comprises a polynucleotide of any one of claims 1-7, an rAAV of claim 7 or 8, a rAAV particle of claim 9 or a composition of claim 10 or 11.

37. The composition of any one of claims 30-36, wherein the composition is administered with an immunosuppressing agent.

38. The composition of any one of claims 30-36, wherein the composition is formulated for intravenous delivery.


SEQUENCE LISTING (7 sequences)

SEQ ID NO: 1 - DNA, 1200 bp, Homo sapiens
atgaaaatgcggttcttggggttggtggtctgtttggttctctggaccctgcattctgaggggtctggagggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtga

SEQ ID NO: 2 - protein, 399 aa, Homo sapiens
MKMRFLGLVVCLVLWTLHSEGSGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 3 - DNA, 228 bp, Homo sapiens
tcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 4 - DNA, 5626 bp, Homo sapiens
acccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgtgtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctttcgttacataacttacggtaaatggcccgcctggctgaccgcccaacgacccccggactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgctcgaccggggtacccggccggctagccgccaccatgaaaatgcggttcttggggttggtggtctgtttggttctctggaccctgcattctgaggggtctggagggaaactgacagctgtggatcctgaaacaaacatgaatgtgagtgaaattatctcttactggggattccctagtgaggaatacctagttgagacagaagatggatatattctgtgccttaaccgaattcctcatgggaggaagaaccattctgacaaaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcagttatgatgagatggcaaaatatgacctaccagcttccattaacttcattctgaataaaactggccaagaacaagtgtattatgtgggtcattctcaaggcaccactataggttttatagcattttcacagatccctgagctggctaaaaggattaaaatgttttttgccctgggtcctgtggcttccgtcgccttctgtactagccctatggccaaattaggacgattaccagatcatctcattaaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatgtctagagtggatgtatatacaacacattctcctgctggaacttctgtgcaaaacatgttacactggagccaggctgttaaattccaaaagtttcaagcctttgactggggaagcagtgccaagaattattttcattacaaccagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtgagcatgcactagtgcggccgcggatctcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaggtttaaaccccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattcgttcttcggggcgaaaactctcaaggatcttaccgctgttgagatccagttcgatgtaacccactcgtgc

SEQ ID NO: 5 - DNA, 192 bp, Homo sapiens
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 6 - DNA, 130 bp, Homo sapiens
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 7 - DNA, 228 bp, Homo sapiens
aaccccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcag